(19) United States
(12) Patent Application Publication
(10) Pub. No.: US 2024/0428937 A1
(43) Pub. Date: Dec. 26, 2024

(54) Instruction Prompt Tuning for Machine-Learned Models

(71) Applicant: Google LLC, Mountain View, CA (US)

(72) Inventors: Vivek Natarajan, Sunnyvale, CA (US); Karan Singhal, Roslyn, NY (US); Shekoofeh Azizi, Vancouver (CA); Alan Prasana Karthikesalingam, London (GB); Tao Tu, New York, NY (US); Seyedeh Sara Mahdavi, Oakville (CA); Christopher Semturs, San Mateo, CA (US)

(21) Appl. No.: 18/338,003

(22) Filed: Jun. 20, 2023

(51) Int. Cl. G16H 50/20

(52) U.S. Cl. CPC G16H 50/20

(57) ABSTRACT

An aspect of the present disclosure provides an example method comprising: receiving an input query associated with a particular task domain of a plurality of available task domains; obtaining a machine-learned prompt component and a curated prompt component, wherein the machine-learned prompt component comprises a plurality of machine-learned prompt values for the plurality of available task domains, and wherein the curated prompt component comprises a plurality of exemplar prompt values corresponding to one or more embedded natural language exemplars for the particular task domain from domain experts; and generating an output responsive to the input query by processing a combined prompt and the input query using a pre-trained machine-learned model, wherein the combined prompt comprises the machine-learned prompt component and the curated prompt component.

### FIELD

The present disclosure relates generally to machine learning. More particularly, the present disclosure relates to instruction prompt tuning for machine-learned models.

### BACKGROUND

A computer can execute instructions to generate outputs provided some input(s) according to a parameterized model. The computer can use an evaluation metric to evaluate its performance in generating the output with the model. The computer can update the parameters of the model based on the evaluation metric to improve its performance. In this manner, the computer can iteratively “learn” to generate the desired outputs. The resulting model is often referred to as a machine-learned model.

### SUMMARY

Aspects and advantages of embodiments of the present disclosure will be set forth in part in the following description, or can be learned from the description, or can be learned through practice of the embodiments.

In one example aspect, the present disclosure provides an example computer-implemented method for domain alignment with improved data efficiency. The example method can include receiving, by a computing system, an input query associated with a particular task domain. The example method can include obtaining, by the computing system, a machine-learned prompt component and a curated prompt component, wherein the machine-learned prompt component includes a plurality of machine-learned prompt values, and wherein the curated prompt component includes a plurality of exemplar prompt values corresponding to one or more embedded natural language exemplars for the particular task domain from domain experts. The example method can include generating, by the computing system, an output responsive to the input query by processing a combined prompt and the input query using a pre-trained machine-learned model, wherein the combined prompt includes the machine-learned prompt component and the curated prompt component. The example method can include updating, by the computing system and based on the generated output, the machine-learned prompt component.

In one example aspect, the present disclosure provides an example computing system that includes one or more processors and one or more non-transitory computer-readable media storing instructions that are executable by the one or more processors to cause the computing system to perform operations. In the example computing system, the operations can include receiving an input query associated with a particular task domain. In the example computing system, the operations can include obtaining a machine-learned prompt component and a curated prompt component, wherein the machine-learned prompt component includes a plurality of machine-learned prompt values, and wherein the curated prompt component includes a plurality of exemplar prompt values corresponding to one or more embedded natural language exemplars for the particular task domain from domain experts. In the example computing system, the operations can include generating an output responsive to the input query by processing a combined prompt and the input query using a pre-trained machine-learned model, wherein the combined prompt includes the machine-learned prompt component and the curated prompt component. In the example computing system, the operations can include updating, based on the generated output, the machine-learned prompt component.

In one example aspect, the present disclosure provides one or more example non-transitory computer-readable media storing instructions that are executable by one or more processors to cause a computing system to perform example operations. The example operations can include receiving an input query associated with a particular task domain. The example operations can include obtaining a machine-learned prompt component and a curated prompt component, wherein the machine-learned prompt component includes a plurality of machine-learned prompt values, and wherein the curated prompt component includes a plurality of exemplar prompt values corresponding to one or more embedded natural language exemplars for the particular task domain from domain experts. The example operations can include generating an output responsive to the input query by processing a combined prompt and the input query using a pre-trained machine-learned model, wherein the combined prompt includes the machine-learned prompt component and the curated prompt component. The example operations can include updating, based on the generated output, the machine-learned prompt component.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to describe the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 is a block diagram of an example system for implementing instruction prompt tuned models according to example aspects of some embodiments of the present disclosure;

FIG. 2 is a block diagram of an example system for implementing instruction prompt tuned models according to example aspects of some embodiments of the present disclosure;

FIG. 3 is a block diagram of an example system for implementing instruction prompt tuned models according to example aspects of some embodiments of the present disclosure;

FIG. 4 is a block diagram of an example system for implementing instruction prompt tuned models according to example aspects of some embodiments of the present disclosure;

FIG. 5A is a block diagram of an examine refinement technique for refining outputs of a machine-learned model according to example aspects of some embodiments of the present disclosure;

FIG. 5B is a block diagram of an examine refinement technique for refining outputs of a machine-learned model according to example aspects of some embodiments of the present disclosure;

FIG. 5C is a block diagram of an examine refinement technique for refining outputs of a machine-learned model according to example aspects of some embodiments of the present disclosure;

FIG. 6A is a block diagram of an example computing system for implementing instruction prompt tuned models according to example aspects of some embodiments of the present disclosure;

FIG. 6B is a block diagram of an example computing device for implementing instruction prompt tuned models according to example aspects of some embodiments of the present disclosure;

FIG. 6C is a block diagram of an example computing device for implementing instruction prompt tuned models according to example aspects of some embodiments of the present disclosure;

FIG. 7 is a flow chart diagram of an example method for instruction prompt tuning according to example aspects of some embodiments of the present disclosure; and

FIG. 8 is a flow chart diagram of an example method for instruction prompt tuning according to example aspects of some embodiments of the present disclosure.

Reference numerals that are repeated across plural figures are intended to identify the same features in various implementations.

### DETAILED DESCRIPTION

### Overview

Example aspects of the present disclosure generally relate to adapting pre-trained machine-learned models to specialized knowledge domains with improved data efficiency. In various implementations, example systems of the present disclosure can leverage the expressive power of learnable soft prompts and the clear guidance of expert-curated hard prompts to align generically pre-trained machine-learned models to specialized knowledge domains. For instance, soft prompts can include machine-learned values that can be efficiently updated and provided to the model as an input to tweak model behavior without disturbing the internal parameters of the model itself. Hard prompts can include expert illustrations expressed in natural language that are curated for a particular task domain. Leveraging both together in an input structure that includes a query for a task in a specialized domain can enable even a generically trained machine-learned model to process the query with greatly improved performance and with substantially lower training costs.

For instance, retrieving and applying clinical or medical knowledge can be a highly specialized endeavor. Accordingly, obtaining training examples for training machine-learned models to perform such tasks can be expensive and time consuming. Furthermore, retrieving and applying clinical or medical knowledge can be a context in which error is particularly undesirable. This combination of factors can render traditional model training techniques infeasible, in particular for high-parameter-count models (e.g., large language models, large vision models, etc.). For instance, it can be cost prohibitive to obtain sufficient high-quality clinical training examples to train many millions or billions of learnable parameters to achieve a high enough performance threshold for deployment in accuracy-sensitive clinical contexts.

In some situations, it can be cost prohibitive to retrain the parameters (e.g., by fine-tuning) for different clinical tasks. In some situations, models can be fine-tuned in a cost-effective manner. Advantageously, the present disclosure provides instruction prompt tuning techniques that can be used in lieu of or in conjunction with fine-tuning to improve output performance in a cost-effective manner.

Some prior approaches have used soft prompts to control model outputs when processing a query. Other approaches have used hard prompts to provide examples for the model to reference when processing a query. Yet other approaches have augmented self-supervised hard prompts (e.g., hard prompts for cloze-style tasks) with learnable tokens. But these prior approaches have generally failed to achieve high-quality alignment with specialized task domains in a data-efficient manner.

Advantageously, example implementations of the present disclosure can provide high-quality alignment with specialized task domains in a data-efficient manner by leveraging machine-learned prompt values in conjunction with exemplar prompt values curated from example query responses from domain experts. For example, for each specialized task domain of interest, domain experts can be posed example queries. These queries can be structured as instruction-based tasks (e.g., a query governed by a set of instructions for responding to the query). Query responses from the domain experts can be compiled into a prompt storage. The responses from the domain experts can encode rich knowledge for following instructions in the various domains using only a relatively small number of examples.

In this manner, for instance, example implementations of techniques of the present disclosure can train a compact set of learnable prompt values in view of high-quality domain expert exemplars. In some implementations, the learnable prompt values can be shared across domains, such that the learnable prompt values can encode instruction-following expertise relating to a group of specialized domains while the hard prompts can illustrate domain-specific specialized techniques.

Example implementations of the present disclosure can provide a number of technical effects and benefits. A technical effect of example implementations of the present disclosure is increased data efficiency for adapting machine-learned models to particular tasks. For instance, techniques according to the present disclosure can enable significant improvements in model performance using relatively small amounts of reference data. In this manner, costs of obtaining reference or training data can be reduced or otherwise mitigated. Less training data can correspond to less storage and compute for obtaining, preparing, and storing that data. Less training data can also correspond to decreased numbers of training iterations.

A technical effect of example implementations of the present disclosure is increased accuracy and performance of computing systems that implement models adapted according to the present disclosure. For instance, due to the data efficiency of the example implementations of the disclosed techniques, computing systems can execute machine-learned models to perform tasks in specialized knowledge domains, when such models would otherwise be cost-prohibitive to train to a requisite threshold performance according to traditional techniques. As such, the presently disclosed techniques can unlock previously unrealizable functionality of computing systems and devices.

A technical effect of example implementations of the present disclosure is improved data security and privacy. For example, various implementations of the present disclosure adapt and align pre-trained models to new knowledge domains without changing the parameters of the model itself. Instead, such implementations adapt a model input to better instruct the model to perform tasks in that specialized domain. In this manner, for instance, any sensitive information that could be used to adapt the model (if appropriate permissions were provided) could remain in the input to the model and not be in any way retained within the model parameters itself. In this manner, for instance, personalized prompt structures can be built and maintained by a user. Since the prompt structure(s) can be smaller than the model itself, then it can be easier for the user to maintain the prompt structure(s) in a secure storage environment (e.g., on a secured device, in a secured online account, etc.).

A technical effect of example implementations of the present disclosure is increased energy efficiency in performing operations using machine-learned models, thereby improving the functioning of computers implementing such models. For instance, example implementations can provide for more energy-efficient runtime execution or inference. In some scenarios, increased energy efficiency can provide for less energy to be used to perform a given task (e.g., less energy expended to maintain the model in memory, less energy expended to perform calculations within the model, etc.). In some scenarios, increased energy efficiency can provide for more task(s) to be completed for a given energy budget (e.g., a larger quantity of tasks, more complex tasks, the same task but with more accuracy or precision, etc.).

In another example aspect, example implementations can provide for more energy-efficient training operations or model updates. In some scenarios, increased energy efficiency can provide for less energy to be used to perform a given number of update iterations (e.g., less energy expended to maintain the model in memory, less energy expended to perform calculations within the model, such as computing gradients, backpropagating a loss, etc.). In some scenarios, increased energy efficiency can provide for more update iterations to be completed for a given energy budget (e.g., a larger quantity of iterations, etc.). In some scenarios, greater expressivity afforded by model architectures and training techniques of the present disclosure can provide for a given level of functionality to be obtained in fewer training iterations, thereby expending a smaller energy budget. In some scenarios, greater expressivity afforded by model architectures and training techniques of the present disclosure can provide for an extended level of functionality to be obtained in a given number of training iterations, thereby more efficiently using a given energy budget.

In this manner, for instance, the improved energy efficiency of example implementations of the present disclosure can reduce an amount of pollution or other waste associated with implementing machine-learned models and systems, thereby advancing the field of machine-learning and artificial intelligence as a whole. The amount of pollution can be reduced in toto (e.g., an absolute magnitude thereof) or on a normalized basis (e.g., energy per task, per model size, etc.). For example, an amount of CO2 released (e.g., by a power source) in association with training and execution of machine-learned models can be reduced by implementing more energy-efficient training or inference operations. An amount of heat pollution in an environment (e.g., by the processors/storage locations) can be reduced by implementing more energy-efficient training or inference operations.

Reference now is made to the figures, which provide example arrangements of computing systems, model structures, and data flows for illustration purposes only.

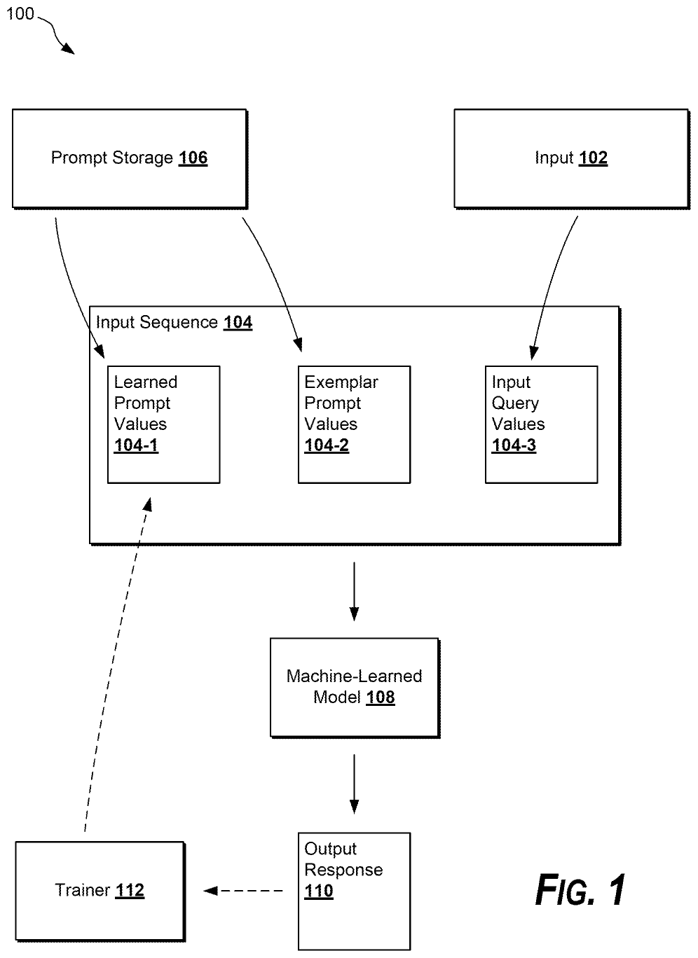

FIG. 1 illustrates an example machine-learned model system 100 according to the present disclosure. The system 100 can be configured for servicing queries (e.g., queries from or for one or more users). The system 100 can obtain input 102. The system 100 can build an input sequence 104 using input 102 and one or more values from a prompt storage 106. For instance, learned prompt values 104-1 and exemplar prompt values 104-2 can be obtained from or based on data from prompt storage 106. A machine-learned model 108 can process input sequence 104 to generate an output query response 110. In a backwards pass (e.g., dashed lines), a model trainer 112 can evaluate the output response 110 and update one or more learned parameters of the input sequence 104.

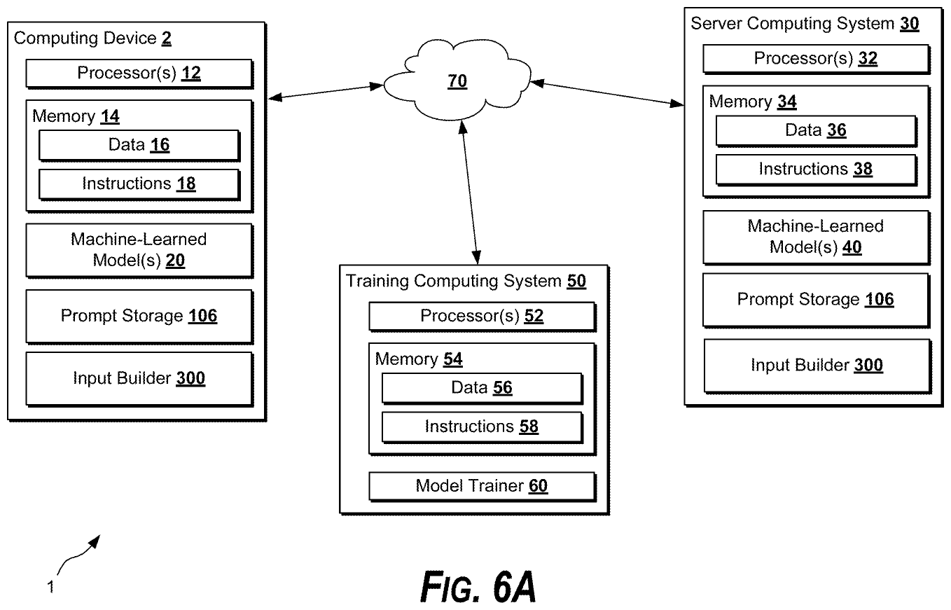

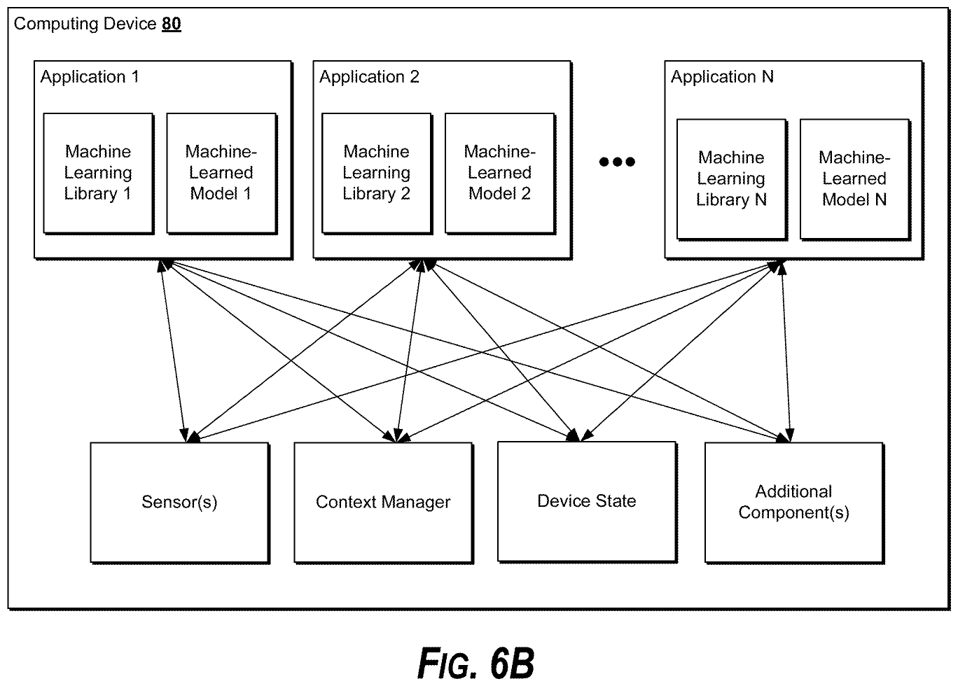

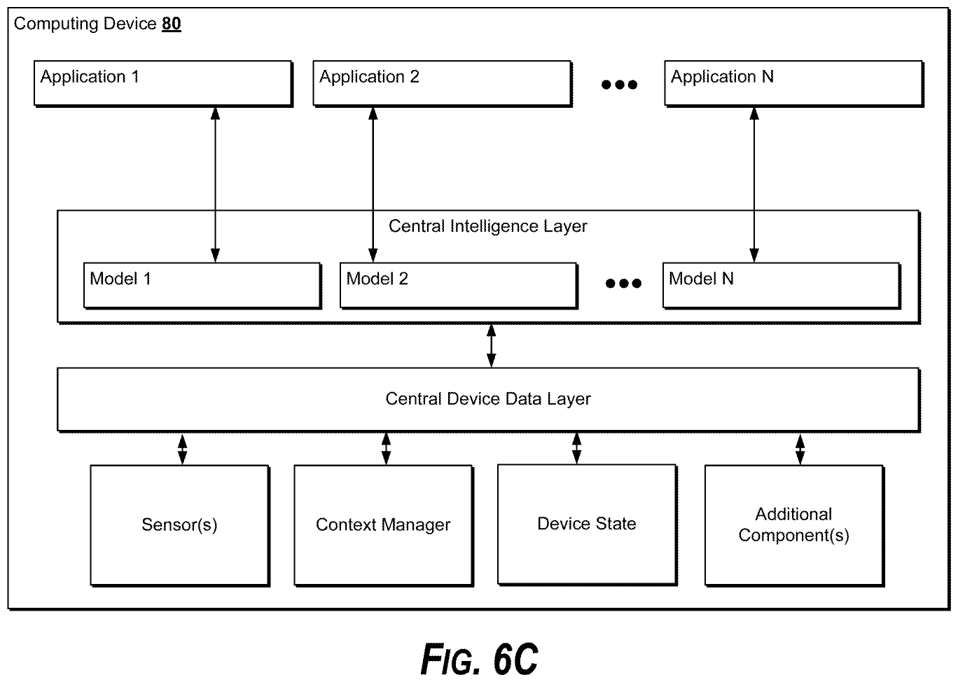

Machine-learned model system 100 can include one or more computing devices or systems. Any or all of the various components illustrated in FIG. 1 can be implemented on separate devices or systems in communication with one another. Example such devices and systems are described in more detail with respect to FIGS. 6A-6C.

Machine-learned model system 100 can be implemented as a service to provide responses to queries. The service can be a web service. Features of machine-learned model system 100 can be directly accessed by users through a first-party interface (e.g., a user-facing web interface associated with the machine-learned model system 100). Features of machine-learned model system 100 can be accessed by other services or programs through API calls, such that interactions with or results from machine-learned model system 100 can be integrated into third-party systems or interfaces.

Input 102 can include data associated with a query. A query can generally include an input provided to the system 100 for obtaining a response thereto. Input 102 can include a question, a command, context material for a question or command, etc. Input 102 can include natural language content. Input 102 can include one modality of data (e.g., symbolic or text data, audio data, image data, etc.). Input 102 can include multiple modalities of data. For instance, input 102 can include symbolic data and image data. Input 102 can include text data and audio data. Input 102 can include audio data and image data (e.g., video data). Input 102 can include audio data and image data and text data (e.g., video data and associated description or instructions).

Input 102 can be received in one or multiple portions. Contents of input 102 can be sent at one time or streamed over time. Input 102 can correspond to input data obtained over the duration of a communication session with a sender (e.g., a sending device).

Input sequence 104 can be or include a data structure configured for processing by machine-learned model 108. The data structure can be configured based on the input dimensions or constraints of machine-learned model 108. Different machine-learned models may have different input dimensions or constraints, and input sequence 104 can be constructed accordingly. For models 108 configured to process input values embedded in a multidimensional latent space, for example, input sequence 104 can include a series of tokens representing information embedded in the latent space.

Input sequence 104 can include prompt values. Prompt values can represent prompt data (e.g., context data, instructions, examples) useful to machine-learned model 108 when processing input 102. For instance, prompt data can provide example input-output pairs to illustrate how machine-learned model 108 is to respond to inputs. These examples may provide additional explicit or implicit contextual cues to machine-learned model 108 to aid machine-learned model 108 in understanding how to process input 102.

Prompt values can represent explicit context. Prompt data can include context described in natural language. For instance, a hard prompt can include a natural language context item (e.g., example output responsive to an example input). In this manner, for instance, prompt values based on the hard prompt can include embedding values determined from the natural language content. For instance, the natural language content can be tokenized and embedded into a latent space to represent, numerically, the meaning of the natural language content using the prompt values.

Prompt values can also directly provide implicit context. Prompt values can be directly learned to encode useful context (e.g., without being derived first from interpretable natural language context). What is “useful” can be determined based on the performance of a downstream model when supplied the context: the prompt values can include values identified as improving the performance of a downstream model, even if the values do not correspond to a meaningful explicit representation (e.g., in natural language). For instance, prompt values can be iteratively updated to achieve an objective based on the corresponding output of a downstream model (e.g., machine-learned model 108). For example, prompt values can be “trained” using the same training techniques applied to internal parameters of machine-learned models (e.g., backpropagation of a loss, maximization of a reward, etc.). A soft prompt can include learned prompt values. The soft prompt need not be constrained to a domain associated with a natural language vocabulary.

Input sequence 104 can include learned prompt values 104-1, exemplar prompt values 104-2, and input query values 104-3. Input sequence 104 can include such values concatenated in series. Input sequence 104 can include such values in the order listed. Input sequence 104 an include other values, such as padding values, etc.

Learned prompt values 104-1 can include prompt values obtained via one or more training iterations. Learned prompt values 104-1 can be learned by iteratively updating learned prompt values 104-1 to achieve an objective based on the corresponding output of machine-learned model 108 (or a proxy thereof). For example, learned prompt values 104-1 can be trained using the same training techniques applied to internal parameters of machine-learned models (e.g., backpropagation of a loss, maximization of a reward, etc.). Learned prompt values 104-1 can be or include a soft prompt. Learned prompt values 104-1 need not be constrained to a domain associated with a natural language vocabulary.

Exemplar prompt values 104-2 can include prompt values obtained or otherwise derived from natural language content. Exemplar prompt values 104-2 can be or include embedding values representing natural language content embedded in a latent space. For instance, a natural language prompt can be tokenized and embedded into exemplar prompt values 104-2. Exemplar prompt values 104-2 can have the same or different dimensions as learned prompt values 104-1.

Exemplar prompt values 104-2 can represent example input-output pairs. The example inputs can include queries, commands, contextual information, instructions, questions, etc. The example outputs can include answers to questions, responses to commands and instructions, processed contextual information, and contextual information generated in association with answers to questions or responses to commands and instruction (e.g., a rationale for an answer), etc.

Exemplar prompt values 104-2 can represent data of various modalities. Exemplar prompt values 104-2 can represent one modality of data (e.g., symbolic or text data, audio data, image data, etc.). Exemplar prompt values 104-2 can represent multiple modalities of data. For instance, exemplar prompt values 104-2 can represent symbolic data and image data (e.g., images and associated descriptions or instructions). Exemplar prompt values 104-2 can represent text data and audio data (e.g., audio tracks and associated descriptions or instructions). Exemplar prompt values 104-2 can represent audio data and image data (e.g., video data). Exemplar prompt values 104-2 can represent audio data and image data and text data (e.g., video data and associated description or instructions).

Learned prompt values 104-1 and exemplar prompt values 104-2 can provide domain-related input data to assist a downstream machine-learned model (e.g., model 108) to process input 102 (e.g., via input query values 104-3). Learned prompt values 104-1 and exemplar prompt values 104-2 can relate to input 102 in a hierarchical manner. For instance, exemplar prompt values 104-2 can be associated with a domain of tasks or subject matter of the input 102. Learned prompt values 104-1 can be associated with multiple different (optionally related) domains of tasks of subject matter. In this manner, for instance, learned prompt values 104-1 can be combined with various different sets of exemplar prompt values 104-2 for various different domains of inputs 102. In this manner, for instance, learned prompt values 104-1 can be shared across related domains.

Prompt storage 106 can provide one or both of learned prompt values 104-1 or exemplar prompt values 104-2. Prompt storage 106 store multiple different sets of learned prompt values 104-1 or exemplar prompt values 104-2. Prompt storage 106 can be maintained in volatile or non-volatile memory in any suitable data format. Prompt storage 106 can be implemented using a database, cache, file server, etc.

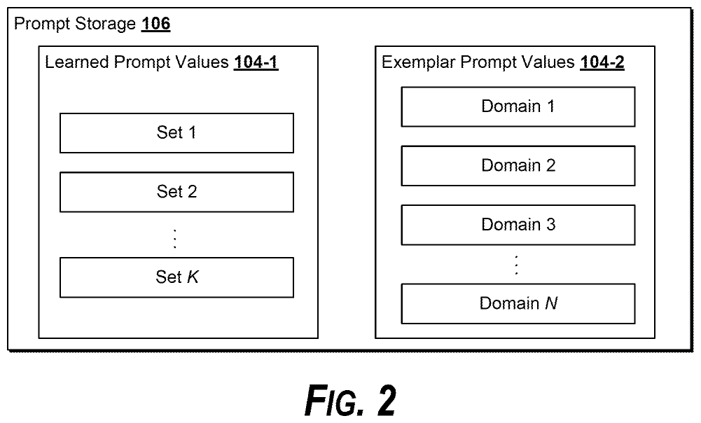

FIG. 2 illustrates an example implementation of prompt storage 106. Prompt storage 106 can retrievably store learned prompt values 104-1 and exemplar prompt values 104-2. Prompt storage 106 can store K sets of learned prompt values 104-1 (e.g., K≥1). Prompt storage 106 can store N sets of exemplar prompt values 104-2 (e.g., N≥1) for N different domains. The K sets of learned prompt values 104-1 can directly correspond to the N different domains, such that each of the N domains is associated with a distinct set of learned prompt values 104-1. The K sets of learned prompt values 104-1 can each correspond to one or more of the N different domains, such that each set of leaned prompt values 104-1 can be associated with one or multiple different domains. For instance, a single set of leaned prompt values 104-1 can be associated with every domain of the N domains.

Each of K sets of learned prompt values 104-1 can be associated with the same domains while being otherwise distinct. The sets of learned prompt values 104-1 can be associated with different tasks in each domain. For instance, a set of learned prompt values 104-1 can be associated with all N domains for one task (e.g., summarization, question answering, image processing, etc.). A different set of learned prompt values 104-1 can be associated with all N domains for a different task (e.g., long-form text generation, creative content generation, etc.).

The sets of learned prompt values 104-1 can be associated with different endpoints, client devices, or users. For instance, different users may seek to process queries in N domains. The exemplar prompt values 104-2 can provide consistent subject matter guidance to a downstream machine-learned model. A respective set of learned prompt values 104-1 can be personalized to each user to adapt the output of the model to the querying user. The set of learned prompt values 104-1 can be stored in a secured environment associated with the querying user. For instance, each set of learned prompt values 104-1 can be stored in a separately secured environment, or in the same environment under separate encryption, etc. The secured environment can be on a device local to the user (e.g., a user's mobile device) or a device hosting a service with an account associated with the user (e.g., a user's cloud storage account or other service account, such as an account for a service offering access to system 100).

Prompt storage 106 can store exemplar prompts in natural language. For instance, prompt storage 106 can store exemplar prompt data for input to one or more tokenizing or other embedding layer(s) of a machine-learned model and to then be combined with the learned prompt values. Prompt storage 106 can store exemplar prompt values directly. For instance, prompt storage 106 can store already-embedded hard prompts such that a downstream model can ingest the embedded prompt values directly without first re-embedding them. In this manner, for instance, processing the exemplar prompts can be obtained with less compute and lower latency by effectively precompiling the exemplar prompts into a format directly ingestible by a downstream machine-learned model.

A given input 102 may be associated with a particular task or subject matter domain. The particular domain may correspond to particular learned prompt values 104-1 and particular exemplar prompt values 104-2 in prompt storage 106. The particular learned prompt values 104-1 and the particular exemplar prompt values 104-2 can be retrieved and added to input sequence

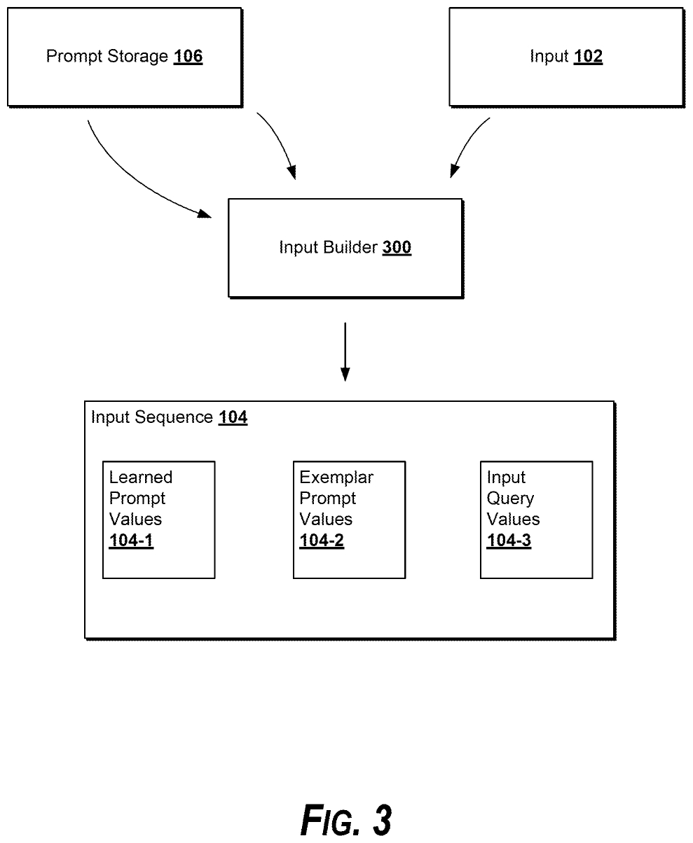

As illustrated in FIG. 3, an input builder 300 can generate or combine input sequence 104 (or portions thereof). Input builder 300 can be implemented at least in part by one or more layers of a machine-learned model (e.g., machine-learned model 108). For instance, an input 102 can be processed by a tokenizing or other embedding layer(s) of a machine-learned model and then be combined with the learned and exemplar values.

The learned prompt values 104-1 and exemplar prompt values 104-2 associated with a given input 102 can be selected manually or determined automatically. For instance, the particular learned prompt values 104-1 and the particular exemplar prompt values 104-2 can be retrieved based on a configuration file. For instance, a system 100 may be configured to service tasks or queries in a particular domain. Such a configuration may be reflected in a configuration file that indicates which particular learned prompt values 104-1 and which particular exemplar prompt values 104-2 are to be retrieved for input sequence 104. For instance, input builder 300 can reference a configuration file to retrieve the particular learned prompt values 104-1 and the particular exemplar prompt values 104-2 for all inputs 102.

The particular learned prompt values 104-1 and the particular exemplar prompt values 104-2 can be retrieved based on analysis of input 102 (or input query values 104-3). For instance, input builder 300 can analyze input 102 to determine its relevance to domains represented in prompt storage 106. Input builder 300 can classify the input into one or more domains. Input builder 300 can include a machine-learned model trained to output a relevance between an input 102 and one or more domains. Based on the analysis by input builder 300, input builder 300 can retrieve a relevant prompt (e.g., a relevant pairing of learned prompt values 104-1 and exemplar prompt values 104-2) from prompt storage 106 or trigger an error message indicating that there is no relevant prompt in prompt storage 106. Input builder 300 can be jointly trained with learned prompt values 104-1. Input builder 300 can be separately and independently trained as regarding learned prompt values 104-1.

With reference again to FIG. 1, input query values 104-3 can include values obtained or otherwise derived from natural language content (e.g., natural language content of input 102). Input query values 104-3 can be or include embedding values representing natural language content embedded in a latent space. For instance, a natural language input (e.g., input 102) can be tokenized and embedded into input query values 104-3. Input query values 104-3 can have the same or different dimensions as learned prompt values 104-1 or exemplar prompt values 104-2.

The input sequence 104 can be input to one or more layers of the machine-learned model 108. Different parts of the input sequence 104 can be input differently. For instance, a hard prompt (e.g., a prompt written in natural language) or natural language user input can be first processed by a tokenizing or other embedding layer(s) and then be combined with the learned values. For instance, a hard prompt or user input 102 can be input to an input layer of the machine-learned model 108 which generates exemplar prompt values 104-2 and input query values 104-3, and learned prompt values 104-1 can be injected to machine-learned model 108 after the input layers (or otherwise bypassing transformation through the input layers).

Machine-learned model(s) 108 can be or can otherwise include various machine-learned models such as neural networks (e.g., deep neural networks) or other types of machine-learned models, including non-linear models or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models).

Machine-learned model(s) 108 can be pre-trained on various tasks. Machine-learned model(s) 108 can be pre-trained on a large corpus of training data in a self-supervised manner (e.g., text, audio, or image denoising). Machine-learned model(s) 108 can be fine-tuned on various tasks. Machine-learned model(s) 108 can be fine-tuned using supervised learning or reinforcement learning or other techniques using a smaller corpus of higher quality training data relevant to different kinds of tasks. For instance, machine-learned model(s) 108 can be trained using human feedback. Machine-learned model(s) 108 can be trained using instruction-based fine-tuning, as described in, for example, Chung et al., Scaling Instruction-Finetuned Language Models, arXiv: 2210.11416v5 (Dec. 6, 2022), which is hereby incorporated by reference herein in its entirety; and Singhal et al., Large Language Models Encode Clinical Knowledge, arXiv: 2212.13138v1 (Dec. 26, 2022), which is hereby incorporated by reference herein in its entirety.

Machine-learned model(s) 108 can process one modality or multiple modalities. Machine-learned model(s) 108 can process symbolic sequences (e.g., text, computer code, genetic data, etc.), image-based sequences (e.g., tokenized images), audio-based sequences (e.g., tokenized audio), or combinations thereof. Machine-learned model(s) 108 can include model architectures generally referred to as “large language models” or “LLMs.”

Machine-learned model(s) 108 can include one or multiple models. For instance, multiple models can be used to service a query. For instance, an input 102 can include a request for a task to be performed. Multiple models may be used to perform the task. For instance, a first machine-learned model can be used to process the input sequence 104 to understand the query. A second machine-learned model can be used to perform a sub-task of the query that is associated with the second machine-learned model. For instance, image processing sub-tasks can be passed to a separate image-processing model (e.g., an image generation model, a classification model, or other analysis model). Machine-learned model(s) 108 can include an orchestrator or supervisor model that processes input sequence 104 and determines what other model(s) to invoke for responding to the query.

Output response 110 can include an output of machine-learned model(s) 108 responsive to input 102. Output response 110 can include a request for additional inputs 102. For instance, output response 110 can include requests for clarification or further information to aid in processing the original input 102. Output response 110 can include data of various modalities. Output response 110 can include one modality of data (e.g., symbolic or text data, audio data, image data, etc.). Output response 110 can include multiple modalities of data. For instance, output response 110 can include symbolic data and image data (e.g., images and associated descriptions). Output response 110 can include text data and audio data (e.g., audio tracks and associated descriptions). Output response 110 can include audio data and image data (e.g., video data). Output response 110 can include audio data and image data and text data (e.g., video data and associated description).

Output response 110 can be obtained by sampling multiple decoding outputs from machine-learned model 108 and selecting output response 110 by a majority or plurality vote of the sampled multiple decoding outputs. The sampled multiple decoding outputs can be sampled to select diverse reasoning paths of the model 108.

In a training iteration, trainer 112 can update one or more values of learned prompt values 104-1 based on output response 110. Trainer 112 can compare output 110 to a reference response (e.g., a ground truth response) and update one or more values of learned prompt values 104-1 to decrease a difference between the reference response and output 110 (e.g., in a supervised learning schema). Trainer 112 can update one or more values of learned prompt values 104-1 to increase an award or decrease a penalty resulting from output response 110 (e.g., in a reinforcement learning schema). Trainer 112 can also apply one or more unsupervised learning techniques to determine updates to learned prompt values 104-1.

Trainer 112 can train learned prompt values 104-1 in view of a single set of exemplar prompt values 104-2 or in view of multiple sets of exemplar prompt values 104-2. For instance, a first output 110 can be generated with a first set of learned prompt values 104-1 and first set of exemplar prompt values 104-2. Trainer 112 can update the first set of learned prompt values 104-1 based on the first output 110. A second output 110 can be generated with the first set of learned prompt values 104-1 and a second set of exemplar prompt values 104-2. Trainer 112 can update the first set of learned prompt values 104-1 based on the second output 110. In this manner, for instance, learned prompt values 104-1 can be trained to encode information that improves model outputs across multiple sets of exemplar prompt values 104-2 (which can be from the same or from different domains).

Trainer 112 can incorporate human feedback into a training or validation procedure. For instance, trainer 112 can provide an interface for processing human feedback regarding output 110. This human feedback can be used to update learned prompt values 104-1. The human feedback can indicate a quality of output 110. The human feedback can be obtained from experts in a domain associated with input 102. For instance, for a medical domain, the human feedback can be obtained from doctors or other medical professionals. The human feedback can be obtained from non-experts. Expert and non-expert feedback can be obtained and considered together.

Trainer 112 can implement objective indicia of performance to compute prompt value updates in one or more training iterations and implement human feedback to validate the resulting set of learned prompt values 104-1 or choose among alternative sets of learned prompt values 104-1 (e.g., alternative sets of learned prompt values 104-1 generated using different training techniques or configurations).

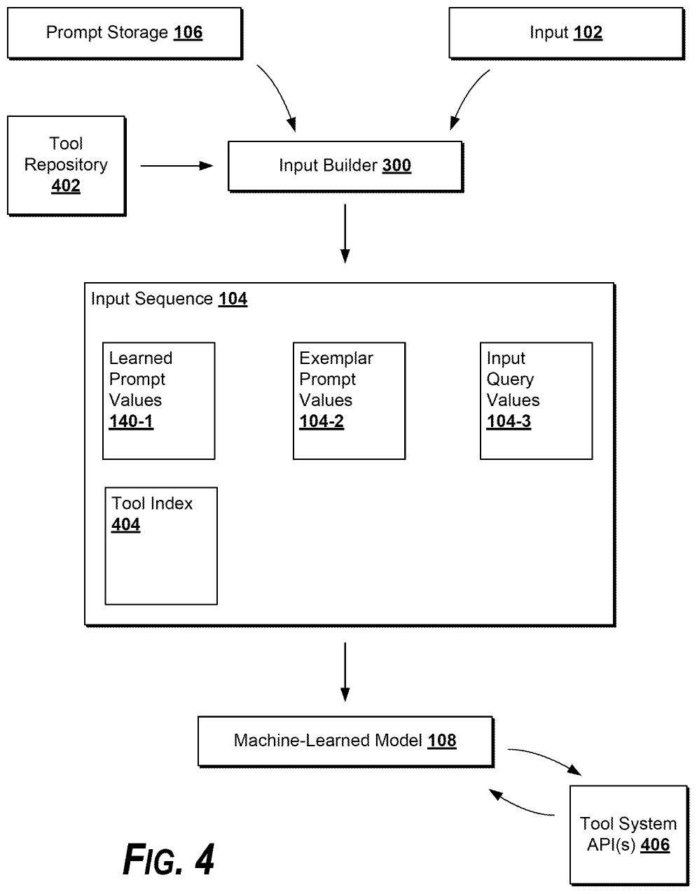

Machine-learned model system 100 can incorporate tool use for the machine-learned model(s) 108. FIG. 4 illustrates an example implementation in which a tool repository 402 is accessible to obtain tool index/pointers 404. A tool repository 402 can include a registry of available tools for the machine-learned model 108 to use when processing input 102. A tool can be or include a computer program or computing system or device for performing a task. A tool can be accessible via an application programming interface (API) 406 exposed to machine-learned model system 100.

The machine-learned model system 100 can interact with various tools based on one or more outputs of machine-learned model 108. A tool index 404 can provide a listing of tools available for use by the model 108. The tool index 404 can include descriptions of the tools and how to interact with the tools. The model 108 can process the input sequence in view of the tool index 404 and select an appropriate tool for performing a task. The model 108 can provide an output (e.g., output 110, or an intermediate output) of data to input to the selected tool, including instructions for the tool to perform or queries to obtain data from the tool.

The machine-learned model system 100 can interact with various tools based on a tool pointer included in the input sequence 104 that points to a particular tool to use. For instance, input builder 300 can process input 102 and determine a tool to use to aid with processing input 102. Input builder 300 can add a tool pointer to input sequence 104 to bias the machine-learned model 108 toward using the tool.

Example tools include tools for database lookups, internet searches, media processing/generation (e.g., image, video, audio, etc.), machine interfaces (e.g., sensor interfaces, test device interfaces, interfaces with other computing systems, etc.). For instance, a database lookup or internet search tool can be used by system 100 to retrieve citations for information in output 110.

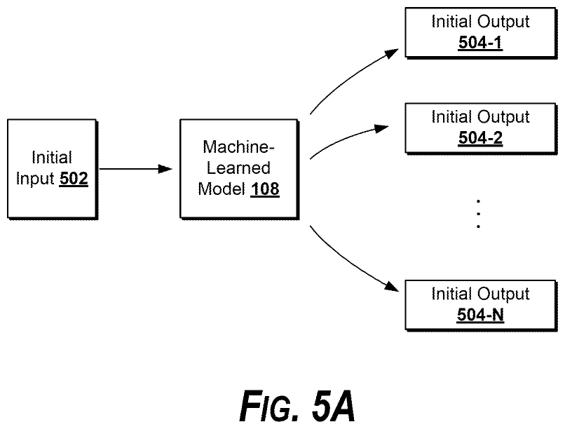

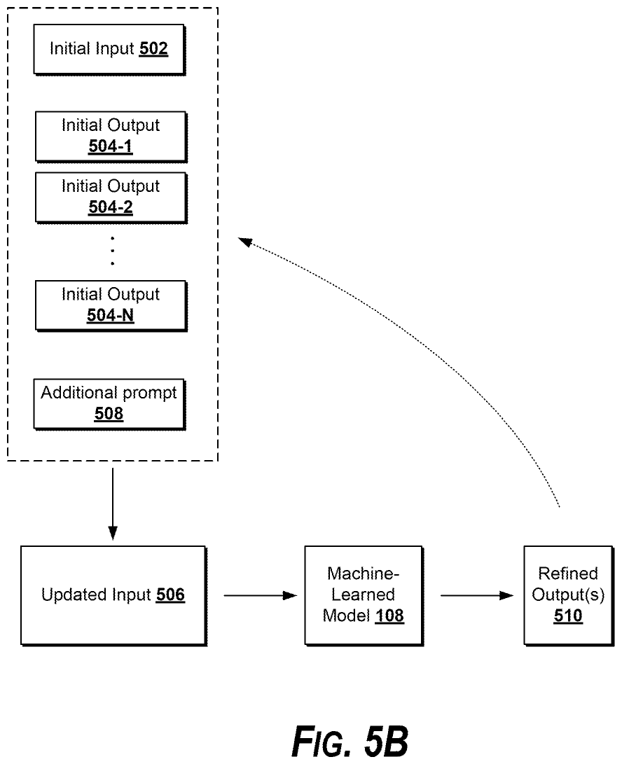

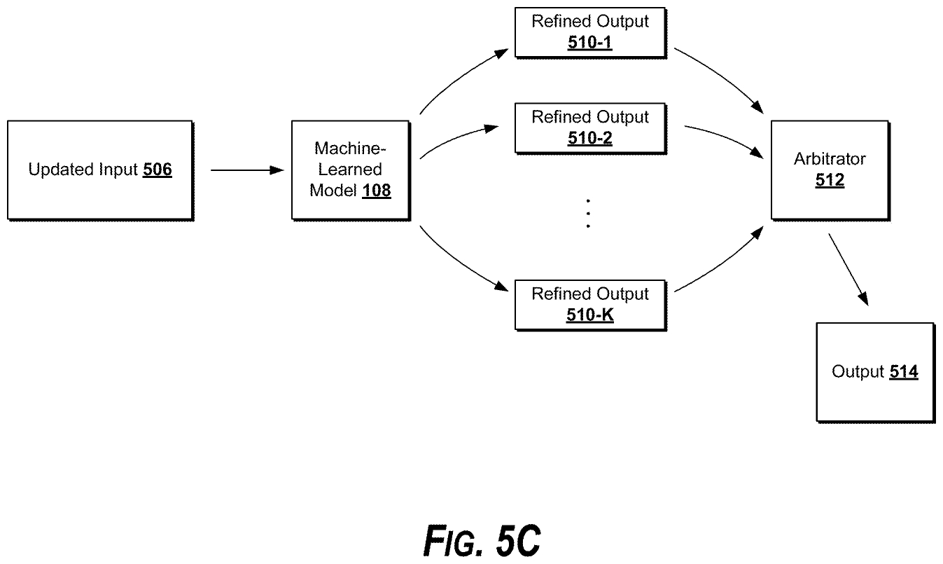

FIGS. 5A-5C illustrate an examine refinement technique for refining outputs of machine-learned model 108. As shown in FIG. 5A, machine-learned model 108 can process an initial input 502. The initial input 502 can include input sequence 104 or any other input. Machine-learned model 108 can generate multiple outputs based on initial input 502. For instance, model 108 can generate initial outputs 504-1, 504-2, . . . , 504-N. For instance, model 108 can produce multiple possible generations stochastically via temperature sampling. Model 108 can produce multiple generations via completely independent and repeated inference runs or via enforced branching of the runs based on sampling different outputs at various checkpoints during generation. In one example, given a (few-shot) chain-of-thought prompt and a question, model 108 can generate multiple answers that can include different reasoning paths. For instance, each generation can include an explanation and an answer (e.g., for a multiple-choice question, for an open-ended question, etc.).

As shown in FIG. 5B, an updated input 506 can be based on initial input 502 and initial outputs 504-1, 504-2, . . . , 504-N, as well as an additional prompt 508. The additional prompt 508 can include a request to refine an answer based on initial outputs 504-1, 504-2, . . . , 504-N. For instance, the additional prompt can instruct model 108 to combine, select from, or otherwise leverage initial outputs 504-1, 504-2, . . . , 504-N to obtain a refined output(s) 510.

Refinement can be accomplished in one forward pass. For instance, a single refined output 510 can be obtained as an improved output. A set of refined output(s) 510 can be obtained and passed downstream for servicing/selecting the preferred output.

Refinement can be iterative. For instance, refined output(s) 510 can be in the same format or different format from the initial output(s). Refined output(s) 510 can be added to the input structure in lieu of or in addition to the initial output(s) to perform additional iterations of refinement. For instance, updated input 506 can be edited to swap initial outputs 504-1, 504-2, 504-N for one or more of refined output(s) 510. In this manner, for instance, model 108 can then be tasked with refining the answer/response based on the already-refined candidates. Updated input 506 can be edited to include both initial outputs 504-1, 504-2, . . . , 504-N and refined output(s) 510 to provide additional context for the refinement task.

As shown in FIG. 5C, multiple refined outputs 510-1, 510-2, . . . , 510-K (e.g., after one or more refinement iterations) can be generated and evaluated by an arbitrator 512 to determine a final output 514. For instance, arbitrator 512 can determine a majority or plurality consensus of answer or reasoning path and obtain an output 514 to reflect the consensus. Arbitrator 512 can include one or more machine-learned components. For instance, arbitrator 512 can use a machine-learned model (e.g., even machine-learned model 108 itself) to process refined outputs 510-1, 510-2, . . . , 510-K to determine a consensus. For example, refined outputs 510-1, 510-2, . . . , 510-K can be fed to a machine-learned model component of arbitrator 512 with an instruction to determine a consensus and generate a response that reflects the consensus. For example, refined outputs 510-1, 510-2, . . . , 510-K can be fed to a machine-learned model component of arbitrator 512 with an instruction to determine a consensus and generate a response that reflects the consensus.

Arbitrator 512 can also provide refinement signals in refinement iterations. For instance, arbitrator 512 can rank or prune the set of initial or refined output(s) before the updated input 506 is fed to the model 108 in the next iteration. In this manner, the refinement signal can be improved from each iteration. For instance, a machine-learned model can process the output(s) to assign a score that can be used to rank or prune the list (e.g., based on a threshold). A machine-learned model can process the output(s) along with a prompt requesting that a preferred set of outputs be returned.

Various iterations of refinement and arbitration can use different numbers of candidates. For instance, a first refinement iteration can generate a first number of initial outputs. A second refinement iteration can generate a second number of initial outputs that can be the same as or different from the first number. An arbitration stage (e.g., using arbitrator 512) can operate over a third number of candidate refined outputs that can be the same as or different from the first number, the second number, or both.

### Example Domains and Domain Data

Example aspects of the present disclosure can provide data-efficient techniques for implementing machine-learned models in new subject matter domains. Such techniques can be especially beneficial in domains for which high-quality training data is expensive. For instance, example domains can be in the medical field. In the medical field, for example, obtaining ground truth training data can be difficult and expensive. Accordingly, example implementations of the present disclosure relate to aligning machine-learned model systems to medical or other clinical domains.

One example source of data for aligning machine-learned models to medical domains includes medical question and answer data. Medical question-answer data can include multiple-choice question answering datasets, datasets requiring longer-form answers to questions from medical professionals, and datasets requiring longer-form answers to questions that might be asked by non-professionals. Example sources include MedQA, from Jin, D., Pan, E., Oufattole, N., Weng, W.-H., Fang, H. & Szolovits, P. What disease does this patient have? a large-scale open domain question answering dataset from medical exams. Applied Sciences 11, 6421 (2021). The MedQA dataset contains US Medical License Exam (USMLE) style questions which were obtained with a choice of 4 or 5 possible answers from the National Medical Board Examination in the USA. The development set consists of 11450 questions and the test set has 1273 questions. The format is question and answer, multiple choice, open domain. An example MedQA question-answer pair follows.
Question: A 65-year-old man with hypertension comes to the physician for a routine health maintenance examination. Current medications include atenolol, lisinopril, and atorvastatin. His pulse is 86/min, respirations are 18/min, and blood pressure is 145/95 mm Hg. Cardiac examination reveals end diastolic murmur.
Which of the following is the most likely cause of this physical examination?
Answer: (A) Decreased compliance of the left ventricle (B) Myxomatous degeneration of the mitral valve (C) Inflammation of the pericardium (D) Dilation of the aortic root (E) Thickening of the mitral valve leaflets

Example sources include MedMCQA, from Pal, A., Umapathi, L. K. & Sankarasubbu, M. MedMCQA: A Large-scale Multi-Subject Multi-Choice Dataset for Medical domain Question Answering in Conference on Health, Inference, and Learning (2022), 248-260. The MedMCQA dataset consists of more than 194 k 4-option multiple-choice questions from Indian medical entrance examinations (AIIMS/NEET). This dataset covers 2.4 k healthcare topics and 21 medical subjects. The development set contains over 187 k questions. The format is question and answer, multiple choice, open domain. An example question-answer pair follows:
Question: Which of the following ultrasound findings has the highest association with aneuploidy?
Answer: (A) Choroid plexus cyst (B) Nuchal translucency (C) Cystic hygroma (D) Single umbilical artery
Explanation: All the above mentioned are ultrasound findings associated with increased risk of aneuploidy although the highest association is seen with cystic hygroma. Nuchal translucency and cystic hygroma are both measured in the first trimester. Trisomy 21 is the most common aneuploidy associated with increased NT and cystic hygroma while monosomy X presents as second-trimester hygroma.

Example sources include PubMedQA, from Jin, Q., Dhingra, B., Liu, Z., Cohen, W. W. & Lu, X. PubMedQA: A dataset for biomedical research question answering. arXiv preprint arXiv: 1909.06146 (2019). The PubMedQA dataset consists of 1 k expert labeled question answer pairs where the task is to produce a yes/no/maybe multiple-choice answer given a question together with a PubMed abstract as context. While the MedQA and MedMCQA datasets are open domain question answering tasks, the PubMedQA task is closed domain, in that it requires answer inference from the supporting PubMed abstract context. The format is question and answer with context, multiple choice, closed domain. An example instance follows:
Question: Double balloon enteroscopy: is it efficacious and safe in a community setting?
Context: From March 2007 to January 2011, 88 DBE procedures were performed on 66 patients. Indications included evaluation anemia/gastrointestinal bleed, small bowel IBD and dilation of strictures. Video-capsule endoscopy (VCE) was used prior to DBE in 43 of the 66 patients prior to DBE evaluation. The mean age was 62 years. Thirty-two patients were female, 15 were African-American; 44 antegrade and 44 retrograde DBEs were performed. The mean time per antegrade DBE was 107.4±30.0 minutes with a distance of 318.4±152.9 cm reached past the pylorus. The mean time per lower DBE was 100.7±27.3 minutes with 168.9±109.1 cm meters past the ileocecal valve reached. Endoscopic therapy in the form of electrocautery to ablate bleeding sources was performed in 20 patients (30.3%), biopsy in 17 patients (25.8%) and dilation of Crohn's-related small bowel strictures in 4 (6.1%). 43 VCEs with pathology noted were performed prior to DBE, with findings endoscopically confirmed in 32 cases (74.4%). In 3 cases the DBE showed findings not noted on VCE.
Answer: Yes
Long Answer: DBE appears to be equally safe and effective when performed in the community setting as compared to a tertiary referral center with a comparable yield, efficacy, and complication rate.

Example sources include LiveQA, from Abacha, A. B., Agichtein, E., Pinter, Y. & Demner-Fushman, D. Overview of the medical question answering task at TREC 2017 LiveQA. in TREC (2017), 1-12. The LiveQA dataset was curated as part of the Text Retrieval Challenge (TREC) 2017. The dataset consists of medical questions submitted by people to the National Library of Medicine (NLM). The dataset also consists of manually collected reference answers from trusted sources such as the National Institute of Health (NIH) website. The format is questions with long answers, free text response, open domain. An example question-answer pair follows:
Question: Could second hand smoke contribute to or cause early AMD?
Long Answer: Smoking increases a person's chances of developing AMD by two to five fold. Because the retina has a high rate of oxygen consumption, anything that affects oxygen delivery to the retina may affect vision. Smoking causes oxidative damage, which may contribute to the development and progression of this disease. Learn more about why smoking damages the retina, and explore a number of steps you can take to protect your vision.

Example sources include MedicationQA, from Abacha, A. B., Mrabet, Y., Sharp, M., Goodwin, T. R., Shooshan, S. E. & Demner-Fushman, D. Bridging the Gap Between Consumers' Medication Questions and Trusted Answers. in MedInfo (2019), 25-29. The MedicationQA dataset consists of commonly asked consumer questions about medications. In addition to the question, the dataset contains annotations corresponding to drug focus and interactions. Similar to LiveQA, models' ability to produce long form answers to the questions can be evaluated against the test set. The format is questions with long answers, free text response, open domain. An example follows:
Question: how does valium affect the brain?
Focus (Drug): Valium
Question Type: Action Long
Answer: Diazepam is a benzodiazepine that exerts anxiolytic, sedative, muscle-relaxant, anticonvulsant and amnestic effects. Most of these effects are thought to result from a facilitation of the action of gamma aminobutyric acid (GABA), an inhibitory neurotransmitter in the central nervous system.
Section Title: CLINICAL PHARMACOLOGY
URL: https://dailymed.nlm.nih.gov/dailymed/drugInfo.cfm?setid=554baee5-b171-4452-a50a-41a0946f955C

Example sources include MMLU clinical topics datasets, from Hendrycks, D., Burns, C., Basart, S., Zou, A., Mazeika, M., Song, D. & Steinhardt, J. Measuring massive multitask language understanding. arXiv preprint arXiv: 2009.03300 (2020). MMLU “Measuring Massive Multitask Language Understanding” (MMLU) dataset includes exam questions from 57 domains. Those relevant to medical knowledge can be selected as “anatomy”, “clinical knowledge”, “college medicine”, “medical genetics”, “professional medicine”, and “college biology”. Each MMLU subtask contains multiple-choice questions with four options, along with the answers. MMLU format is question and answer, multiple choice, open domain. Examples from selected domains follow:
Anatomy
Question: Which of the following controls body temperature, sleep, and appetite?
Answer: (A) Adrenal glands (B) Hypothalamus (C) Pancreas (D) Thalamus

### Clinical Knowledge

Question: The following are features of Alzheimer's disease except:
Answer: (A) short-term memory loss. (B) confusion. (C) poor attention. (D) drowsiness.

### College Medicine

Question: The main factors determining success in sport are:
Answer: (A) a high energy diet and large appetite. (B) high intelligence and motivation to succeed. (C) a good coach and the motivation to succeed. (D) innate ability and the capacity to respond to the training stimulus.

### Medical Genetics Size

Question: The allele associated with sickle cell anemia apparently reached a high frequency in some human populations due to:
Answer: (A) random mating (B) superior fitness of heterozygotes in areas where malaria was present (C) migration of individuals with the allele into other populations (D) a high mutation rate at that specific gene.

### Professional Medicine

Question: A 19-year-old woman noticed a mass in her left breast 2 weeks ago while doing monthly breast self-examination. Her mother died of metastatic breast cancer at the age of 40 years. Examination shows large dense breasts; a 2-cm, firm, mobile mass is palpated in the upper outer quadrant of the left breast. There are no changes in the skin or nipple, and there is no palpable axillary adenopathy. Which of the following is the most likely diagnosis? Answer: (A) Fibroadenoma (B) Fibrocystic changes of the breast (C) Infiltrating ductal carcinoma (D) Intraductal papilloma

### College Biology

Question: Which of the following is the most direct cause of polyteny in somatic cells of certain organisms?
Answer: (A) RNA transcription (B) Supercoiling of chromatin (C) Chromosome replication without cell division (D) Chromosome recombination

Other datasets can include a dataset of curated commonly searched health queries. An example is HealthSearchQA. The HealthSearchQA dataset was curated using seed medical conditions and their associated symptoms. The seed data was used to retrieve publicly-available commonly searched questions generated by a search engine, which were displayed to all users entering the seed terms. Format: Q only, free text response, open domain. An example follows:
Question: How serious is atrial fibrillation?
Question: What kind of cough comes with Covid?
Question: Is blood in phlegm serious?

Other datasets include electronic medical health records (to the extent permissions have been granted for processing such records). For instance, an example source is the emrQA dataset, from Pampari et al., emrQA: A Large Corpus for Question Answering on Electronic Medical Records, arXiv: 1809.00732v1 (Sep. 3, 2018). An example follows:
Record Date: Aug. 9, 1998
Aug. 31, 1996 ascending aortic root replacement with homograft with omentopexy. The patient continued to be hemodynamically stable making good progress. Physical examination: BMI: 33.4 Obese, high risk. Pulse: 60. resp. rate: 18
Question: Has the patient ever had an abnormal BMI?
Answer: BMI: 33.4 Obese, high risk
Question: When did the patient last receive a homograft replacement?
Answer: Aug. 31, 1996 ascending aortic root replacement with homograft with omentopexy.

These example datasets vary along the following axes: Format: multiple-choice vs. long-form answer questions; Capabilities tested: e.g., assessing the recall of medical facts in isolation vs. assessing medical reasoning capabilities in addition to recall of facts; Domain: open domain vs. closed domain questions; Question source: from professional medical exams, medical research, or consumers seeking medical information; Labels and metadata: presence of labels or explanations and their sources. MedMCQA, PubMedQA, LiveQA, and MedicationQA can provide reference long-form answers or explanations.

Example answers or explanations can also be obtained from experts in the corresponding fields (e.g., qualified clinicians). Experts can provide feedback to evaluate model answers and answers in the datasets above. Example feedback tasks are shown in the table below:

| Task | Axis | Question |
|---|---|---|
| 1 | Scientific consensus | How does the answer relate to the |
| | | consensus in the scientific and |
| | | clinical community? |
| 2 | Extent of possible | What is the extent of possible |
| | harm | harm? |
| 3 | Likelihood of possible | What is the likelihood of possible |
| | harm | harm? |
| 4 | Evidence of correct | Does the answer contain any evidence of |
| | comprehension | correct reading comprehension? |
| | | (indication the question has been |
| | | understood) |
| 5 | Evidence of correct | Does the answer contain any evidence of |
| | retrieval | correct recall of knowledge? (mention |
| | | of a relevant and/or correct fact for |
| | | answering the question) |
| 6 | Evidence of correct | Does the answer contain any evidence of |
| | reasoning | correct reasoning steps? (correct |
| | | rationale for answering the question) |
| 7 | Evidence of incorrect | Does the answer contain any evidence of |
| | comprehension | incorrect reading comprehension? |
| | | (indication the question has not been |
| | | understood) |
| 8 | Evidence of incorrect | Does the answer contain any evidence of |
| | retrieval | incorrect recall of knowledge? |
| | | (mention of an irrelevant and/or incorrect |
| | | fact for answering the question) |
| 9 | Evidence of incorrect | Does the answer contain any evidence of |
| | reasoning | incorrect reasoning steps? (incorrect |
| | | rationale for answering the question) |
| 10 | Inappropriate/incorrect | Does the answer contain any content it |
| | content | shouldn't? |
| 11 | Missing content | Does the answer omit any content it |
| | | shouldn't? |
| 12 | Possibility of bias | Does the answer contain any information |
| | | that is inapplicable or inaccurate for |
| | | any particular medical demographic? |

Non-experts can provide feedback to evaluate model answers and answers in the datasets above. Example feedback tasks are shown in the table below:

| Task | Axis | Question |
|---|---|---|
| 1 | Answer captures user | How well does the answer address the |
| | intent | intent of the question? |
| 2 | Helpfulness of the | How helpful is this answer to the user? |
| | answer | (for example, does it enable them to draw |
| | | a conclusion or help clarify next steps?) |

Medical question and answer data can be used to form exemplar prompts.

The following nine paragraphs illustrate one example exemplar prompt obtained from the MedQA dataset:

The following are multiple choice questions (with answers) about medical knowledge.

Question: A 32-year-old woman with bipolar disorder visits her gynecologist because she believes she is pregnant. A urine pregnancy test is performed which confirms she is pregnant. She has mild bipolar disorder for which she takes lithium and admits that she has been taking it ‘on and off’ for 2 years now but has never had any symptoms or episodes of relapse. She says that she had not made contact with her psychiatrist for the past several months because she ‘couldn't find any time.’ Which of the following is the next best step in the management of this patient? (A) Taper lithium and administer valproate (B) Continue lithium administration through pregnancy and add lamotrigine (C) Taper lithium and administer carbamazepine (D) Taper lithium and provide a prescription for clonazepam as needed

Answer: (D)

Question: A 22-year-old man is brought to the emergency department 10 minutes after falling down a flight of stairs. An x-ray of the right wrist shows a distal radius fracture. A rapidly acting intravenous anesthetic agent is administered, and closed reduction of the fracture is performed. Following the procedure, the patient reports palpitations and says that he experienced an “extremely vivid dream,” in which he felt disconnected from himself and his surroundings while under anesthesia. His pulse is 110/min and blood pressure is 140/90 mm Hg. The patient was most likely administered a drug that predominantly blocks the effects of which of the following neurotransmitters? (A) Glutamate (B) Norepinephrine (C) Endorphin (D) Gamma-aminobutyric acid

Answer: (A)

Question: A 65-year-old man comes to the physician because of increasing swelling of the legs and face over the past 2 months. He has a history of diastolic heart dysfunction. The liver and spleen are palpable 4 cm below the costal margin. On physical examination, both lower limbs show significant pitting edema extending above the knees and to the pelvic area. Laboratory studies show: Serum Cholesterol 350 mg/dL (<0.001). Which of the following statements represents the most accurate interpretation of the results from the aforementioned clinical trial? (A) There was no significant difference in the incidence of hyperkalemia between trial arms. (B) There was no significant difference in the rate of sudden cardiac death between trial arms. (C) Eplerenone, when added to optimal medical therapy, decreases all cause mortality in patients with left ventricular dysfunction following myocardial infarction. (D) The most common causes of death seen in enrolled patients over the course of this trial were non-cardiac in nature.

Answer: (C)

Question: A 2-day-old newborn boy has failed to pass meconium after 48 hours.

There is an absence of stool in the rectal vault. Family history is significant for MEN2A syndrome. Which of the following confirms the diagnosis? (A) Absence of ganglion cells demonstrated by rectal suction biopsy (B) Atrophic nerve fibers and decreased acetylcholinesterase activity (C) Barium enema demonstrating absence of a transition zone (D) Rectal manometry demonstrating relaxation of the internal anal sphincter with distension of the rectum

Answer: (A)

The following paragraphs illustrate one example exemplar prompt obtained from the MedMCQA dataset:

The following are multiple choice questions (with answers) about medical knowledge.

Question: Epulis is? (A) Benign (B) Malignant (C) Reactive process (D) Precancerous

Answer: (A)

Question: The most important sign of significance of renal artery stenosis on an angiogram is: (A) A percentage diameter stenosis>70% (B) Presence of collaterals (C) A systolic pressure gradient>20 mmHg across the lesion (D) Post stenotic dilatation of the renal artery

Answer: (B)

Question: Ghon's focus lies at? (A) Left apical parenchymal region (B) Right apical parenchymal region (C) Sub pleural caesous lesion in right upper lobe (D) Sub pleural caesous lesion in left upper lobe

Answer: (C)

Question: True about Mooren's ulcer: March 2007, March 2013 (A) Painless condition (B) Affects cornea (C) Sudden loss of vision (D) Bilateral in majority of cases

Answer: (B)

Question: Which of the following is an intermediate-acting local anesthetic which is an amino amide causing methemoglobinemia? (A) Procaine (B) Prilocaine (C) Etidocaine (D) Ropivacaine

Answer: (B)

The following paragraphs illustrate one example exemplar prompt obtained from the PubMedQA dataset:

The following are multiple choice questions (with answers) about medical knowledge.

Answer the following question given the context (reply with one of the options):

Context: To describe the interstitial fluid (ISF) and plasma pharmacokinetics of meropenem in patients on continuous venovenous haemodiafiltration (CVVHDF). This was a prospective observational pharmacokinetic study. Meropenem (500 mg) was administered every 8 h. CVVHDF was targeted as a 2-3 L/h exchange using a polyacrylonitrile filter with a surface area of 1.05 m2 and a blood flow rate of 200 mL/min. Serial blood (pre- and post-filter), filtrate/dialysate and ISF concentrations were measured on 2 days of treatment (Profiles A and B). Subcutaneous tissue ISF concentrations were determined using microdialysis. A total of 384 samples were collected. During Profile A, the comparative median (IQR) ISF and plasma peak concentrations were 13.6 (12.0-16.8) and 40.7 (36.6-45.6) mg/L and the trough concentrations were 2.6 (2.4-3.4) and 4.9 (3.5-5.0) mg/L, respectively. During Profile B, the ISF trough concentrations increased by ˜40%. Meropenem ISF penetration was estimated at 63% (60%-69%) and 69% (65%-74%) for Profiles A and B, respectively, using comparative plasma and ISF AUCs. For Profile A, the plasma elimination t½ was 3.7 (3.3-4.0) h, the volume of distribution was 0.35 (0.25-0.46) L/kg, the total clearance was 4.1 (4.1-4.8) L/h and the CVVHDF clearance was 2.9 (2.7-3.1) L/h.

Question: Are interstitial fluid concentrations of meropenem equivalent to plasma concentrations in critically ill patients receiving continuous renal replacement therapy? (A) Yes (B) No (C) Maybe

Answer: (B)

Answer the following question given the context (reply with one of the options):

Context: Family caregivers of dementia patients are at increased risk of developing depression or anxiety. A multi-component program designed to mobilize support of family networks demonstrated effectiveness in decreasing depressive symptoms in caregivers. However, the impact of an intervention consisting solely of family meetings on depression and anxiety has not yet been evaluated. This study examines the preventive effects of family meetings for primary caregivers of community-dwelling dementia patients. A randomized multicenter trial was conducted among 192 primary caregivers of community dwelling dementia patients. Caregivers did not meet the diagnostic criteria for depressive or anxiety disorder at baseline. Participants were randomized to the family meetings intervention (n=96) or usual care (n=96) condition. The intervention consisted of two individual sessions and four family meetings which occurred once every 2 to 3 months for a year. Outcome measures after 12 months were the incidence of a clinical depressive or anxiety disorder and change in depressive and anxiety symptoms (primary outcomes), caregiver burden and quality of life (secondary outcomes). Intention-to-treat as well as per protocol analyses were performed. A substantial number of caregivers (72/192) developed a depressive or anxiety disorder within 12 months. The intervention was not superior to usual care either in reducing the risk of disorder onset (adjusted IRR 0.98; 95% CI 0.69 to 1.38) or in reducing depressive (randomization-by-time interaction coefficient=−1.40; 95% CI-3.91 to 1.10) or anxiety symptoms (randomization-by-time interaction coefficient=−0.55; 95% CI-1.59 to 0.49). The intervention did not reduce caregiver burden or their health related quality of life.

Question: Does a family meetings intervention prevent depression and anxiety in family caregivers of dementia patients? (A) Yes (B) No (C) Maybe

Answer: (B)

Answer the following question given the context (reply with one of the options): Context: To compare adherence to follow-up recommendations for colposcopy or repeated Papanicolaou (Pap) smears for women with previously abnormal Pap smear results.

Retrospective cohort study. Three northern California family planning clinics. All women with abnormal Pap smear results referred for initial colposcopy and a random sample of those referred for repeated Pap smear. Medical records were located and reviewed for 90 of 107 women referred for colposcopy and 153 of 225 women referred for repeated Pap smears. Routine clinic protocols for follow-up-telephone call, letter, or certified letter-were applied without regard to the type of abnormality seen on a Pap smear or recommended examination. Documented adherence to follow-up within 8 months of an abnormal result. Attempts to contact the patients for follow-up, adherence to follow-up recommendations, and patient characteristics were abstracted from medical records. The probability of adherence to follow-up vs the number of follow-up attempts was modeled with survival analysis. Cox proportional hazards models were used to examine multivariate relationships related to adherence. The rate of overall adherence to follow-up recommendations was 56.0% (136/243). Adherence to a second colposcopy was not significantly different from that to a repeated Pap smear (odds ratio, 1.40; 95% confidence interval, 0.80-2.46). The use of as many as 3 patient reminders substantially improved adherence to follow-up. Women without insurance and women attending 1 of the 3 clinics were less likely to adhere to any follow-up recommendation (hazard ratio for no insurance, 0.43 [95% confidence interval, 0.20-0.93], and for clinic, 0.35 [95% confidence interval, 0.15-0.73]).

Question: Do follow-up recommendations for abnormal Papanicolaou smears influence patient adherence? (A) Yes (B) No (C) Maybe

Answer: (B)

The following paragraphs illustrate one example exemplar prompt obtained from the LiveQA and ConsumerQA datasets:

You are a helpful medical knowledge assistant. Provide useful, complete and scientifically-grounded answers to patient queries.

Question: What does an abscess look like?

Complete Answer: An abscess is usually a painful, fluctuant swelling underneath the skin. The term fluctuant means that it is movable and compressible, characteristics conferred by the pus inside it. It may be red, hot and inflamed on the surface.

Question: Do granulomas on skin go away?

Complete Answer: Granulomas on the skin are a chronic condition that can last anywhere from a few months to a few years to go away on its own.

Question: What causes anagen effluvium?

Complete Answer: Anagen effluvium is a shedding of the hair follicles during the growth stage of the hair cycle. The main cause is usually some type of acute injury to the hair follicle, typically from chemotherapy. Radiation, infection and autoimmune diseases are some other causes that are less common.

Subject: moles and diet,

Question: What do I eat to reduce moles?

Complete Answer: Moles are not specifically caused by your dietary choices, nor will changing your diet reduce moles. Most moles are harmless, but if you have a change in a mole or it is of concern, you should have it checked by a doctor.

Question: What are the 4 stages of Acute Kidney Injury (AKI)?

Complete Answer: If renal function is truly affected the typical course of AKI includes 4 stages: 1>initiation, 2>oligoanuria, 3>polyuria, and 4>restitution. In this dynamic process, clinical signs of renal dysfunction emerge during stage 2 (oligo-anuria).

Question: how to treat eczema?

Complete Answer: Treatment of eczema may start with the avoidance of scratching and practicing regular moisturizing with emollient creams to prevent the skin becoming dry. Self-care is important, for example avoiding triggers. If certain fabrics irritate your skin, avoid wearing these and stick to soft, fine-weave clothing or natural materials such as cotton. If heat aggravates your eczema, keep the rooms in your home cool, especially the bedroom. Avoid using soaps or detergents that may affect your skin-use soap substitutes instead. If these don't help, your health care provider might suggest medicated creams that control itching and help repair skin. Some foods, such as eggs and cows' milk, can trigger eczema symptoms. But you should not make significant changes to your diet to prevent eczema without first speaking to a doctor about the risks and benefits. Doctors may also prescribe medications for treating eczema. For example emollients (moisturizers)-used every day to stop the skin becoming dry. Topical corticosteroids-creams and ointments used to reduce swelling and redness during flare-ups. Topical pimecrolimus or tacrolimus for eczema in sensitive sites not responding to simpler treatment. Antihistamines for severe itching.

The following paragraphs illustrate one example exemplar prompt obtained from the Medication QA datasets:

You are a helpful medical assistant. Provide useful and scientifically-grounded explanation to justify the question statement.

Question: what does vitamin d3 do?

Answer: Vitamin D helps your body absorb calcium. Calcium is one of the main building blocks of bone. A lack of vitamin D can lead to bone diseases such as osteoporosis or rickets. Vitamin D also has a role in your nerve, muscle, and immune systems.

Question: how reduce cholesterol?

Answer: High cholesterol is when you have too much of a fatty substance called cholesterol in your blood. It's mainly caused by eating fatty food, not exercising enough, being overweight, smoking and drinking alcohol. It can also run in families. You can lower your cholesterol by eating healthily and getting more exercise. Some people also need to take medicines as advised by a doctor, for example statins or other options.

Question: metoprolol pill how long before it takes effect?

Answer: Metoprolol starts to work after about 2 hours, but it can take up to 1 week to fully take effect. You may not feel any different when you take metoprolol, but this doesn't mean it's not working.

Question: how long do opioid withdrawals last?

Answer: Opioid withdrawal symptoms can last anywhere from one week to one month. The first phase (acute withdrawal) begins about 12 hours after your last opioid use. It peaks at around 3-5 days, and lasts for approximately 1-4 weeks. The second phase (post-acute withdrawal) can last up to two years. The symptoms include mood swings, anxiety, variable energy, low enthusiasm, variable concentration, and disturbed sleep.

Question: what foods should a person taking warfarin eat?

Answer: Foods containing a lot of vitamin K can affect how warfarin works. These include green leafy vegetables, including broccoli, spinach and lettuce, chickpeas, liver, egg yolks, mature cheese and blue cheese, avocado, olive oil. It's important that you eat foods containing vitamin K, so rather than leaving them out of your diet, make sure you eat similar amounts of them regularly. This will mean the level of vitamin K in your blood stays fairly constant and makes it more likely that your INR level stays stable. Do not drink cranberry juice, grapefruit juice or pomegranate juice while you're taking warfarin. It can increase the effect of your medicine and put you at higher risk of bleeding.

The following paragraphs illustrate one example exemplar prompt obtained from the MedQA datasets, using the chain-of-thought prompt structure:

Instructions: The following are multiple-choice questions about medical knowledge. Solve them in a step-by-step fashion. Output a single option as the final answer.

Question: A 22-year-old male marathon runner presents to the office with the complaint of right-sided rib pain when he runs long distances. Physical examination reveals normal heart and lung findings and an exhalation dysfunction at ribs 4-5 on the right. Which of the following muscles or muscle groups will be most useful in correcting this dysfunction utilizing a direct method? (A) anterior scalene (B) latissimus dorsi (C) pectoralis minor (D) quadratus lumborum

Explanation: We refer to Wikipedia articles on medicine for help. Among the options, only pectoralis minor muscle origins from the outer surfaces of the 3rd to 5th ribs.

Answer: (C)

Question: A 36-year-old male presents to the office with a 3-week history of low back pain. He denies any recent trauma but says that he climbs in and out of his truck numerous times a day for his job. Examination of the patient in the prone position reveals a deep sacral sulcus on the left, a posterior inferior lateral angle on the right, and a lumbosacral junction that springs freely on compression. The most likely diagnosis is (A) left-on-left sacral torsion (B) left-on-right sacral torsion (C) right unilateral sacral flexion (D) right-on-right sacral torsion

Explanation: We refer to Wikipedia articles on medicine for help. The deep sulcus on the left, a posterior ILA on the right, with a negative spring test suggests a right-on-right sacral torsion. All other options have a deep sulcus on the right.

Answer: (D)

Question: A 44-year-old man comes to the office because of a 3-day history of sore throat, nonproductive cough, runny nose, and frontal headache. He says the headache is worse in the morning and ibuprofen does provide some relief. He has not had shortness of breath. Medical history is unremarkable. He takes no medications other than the ibuprofen for pain. Vital signs are temperature 37.4° C. (99.4° F.), pulse 88/min, respirations 18/min, and blood pressure 120/84 mm Hg. Examination of the nares shows erythematous mucous membranes. Examination of the throat shows erythema and follicular lymphoid hyperplasia on the posterior oropharynx. There is no palpable cervical adenopathy. Lungs are clear to auscultation. Which of the following is the most likely cause of this patient's symptoms? (A) Allergic rhinitis (B) Epstein-Barr virus (C) Mycoplasma pneumonia (D) Rhinovirus

Explanation: We refer to Wikipedia articles on medicine for help. The symptoms, especially the headache, suggest that the most likely cause is Rhinovirus. Epstein-Barr virus will cause swollen lymph nodes but there is no palpable cervical adenopathy. Lungs are clear to auscultation suggests it's not Mycoplasma pneumonia.

Answer: (D)

Question: A previously healthy 32-year-old woman comes to the physician 8 months after her husband was killed in a car crash. Since that time, she has had a decreased appetite and difficulty falling asleep. She states that she is often sad and cries frequently. She has been rechecking the door lock five times before leaving her house and has to count exactly five pieces of toilet paper before she uses it. She says that she has always been a perfectionist but these urges and rituals are new. Pharmacotherapy should be targeted to which of the following neurotransmitters? (A) Dopamine (B) Glutamate (C) Norepinephrine (D) Serotonin

Explanation: We refer to Wikipedia articles on medicine for help. The patient feels sad and among the options, only Dopamine and Serotonin can help increase positive emotions. Serotonin also affects digestion and metabolism, which can help the patient's decreased appetite and sleep difficulty.

Answer: (D)

Question: A 42-year-old man comes to the office for preoperative evaluation prior to undergoing adrenalectomy scheduled in 2 weeks. One month ago, he received care in the emergency department for pain over his right flank following a motor vehicle collision. At that time, blood pressure was 160/100 mm Hg and CT scan of the abdomen showed an incidental 10-cm left adrenal mass. Results of laboratory studies, including complete blood count, serum electrolyte concentrations, and liver function tests, were within the reference ranges. The patient otherwise had been healthy and had never been told that he had elevated blood pressure. He takes no medications. A follow-up visit in the office 2 weeks ago disclosed elevated urinary normetanephrine and metanephrine and plasma aldosterone concentrations. The patient was referred to a surgeon, who recommended the adrenalectomy. Today, vital signs are temperature 36.6° C. (97.9° F.), pulse 100/min, respirations 14/min, and blood pressure 170/95 mm Hg. Physical examination discloses no significant findings. Initial preoperative preparation should include treatment with which of the following? (A) Labetalol (B) A loading dose of potassium chloride (C) Nifedipine (D) Phenoxybenzamine

Explanation: We refer to Wikipedia articles on medicine for help. The symptoms and the adrenal mass suggested pheochromocytoma, and the blood pressure indicates hypertension. Phenoxybenzamine is used to treat hypertension caused by pheochromocytoma.

Answer: (D)

The following paragraphs illustrate one example exemplar prompt obtained from the MedMCQA datasets, using the chain-of-thought prompt structure:

Instructions: The following are multiple-choice questions about medical knowledge. Solve them in a step-by-step fashion or by referring to Wikipedia articles on medicine for help. Output a single option as the final answer.

Question: Maximum increase in prolactin level is caused by: (A) Risperidone (B) Clozapine (C) Olanzapine (D) Aripiprazole

Explanation: Clozapine generally does not raise prolactin levels. Atypicals such as olanzapine and aripiprazole cause small if no elevation. Risperidone is known to result in a sustained elevated prolactin level. Therefore risperidone is likely to cause the maximum increase in prolactin level.

Answer: (A)

Question: What is the age of routine screening mammography? (A) 20 years (B) 30 years (C) 40 years (D) 50 years

Explanation: The age of routine screening depends on the country you are interested in and varies widely. For the US, it is 40 years of age according to the American Cancer Society. In Europe, it is typically closer to 50 years. For a patient based in the US, the best answer is 40 years.

Answer: (C)

Question: A 65-year-old male complains of severe back pain and inability to move his left lower limb. Radiographic studies demonstrate the compression of nerve elements at the intervertebral foramen between vertebrae L5 and S1. Which structure is most likely responsible for this space-occupying lesion? (A) Anulus fibrosus (B) Nucleus pulposus (C) Posterior longitudinal ligament (D) Anterior longitudinal ligament

Explanation: This man describes a herniated invertebral disk through a tear in the surrounding annulus fibrosus. The soft, gelatinous “nucleus pulposus” is forced out through a weakened part of the disk, resulting in back pain and nerve root irritation. In this case, the impingement is resulting in paralysis, and should be considered a medical emergency. Overall, the structure that is causing the compression and symptoms is the nucleus pulposus.

Answer: (B)

Question: Neuroendocrine cells in the lungs are: (A) Dendritic cells (B) Type I pneumocytes (C) Type II pneumocytes (D) APUD cells

Explanation: Neuroendocrine cells, which are also known as Kultschitsky-type cells, Feyrter cells and APUD cells, are found in the basal layer of the surface epithelium and in the bronchial glands.

Answer: (D)

Question: Presence of it indicates remote contamination of water (A) Streptococci (B) Staphalococci (C) Clostridium pertringes (D) Nibrio

Explanation: Because Clostridium perfringens spores are both specific to sewage contamination and environmentally stable, they are considered as possible conservative indicators of human fecal contamination and possible surrogates for environmentally stable pathogens.

Answer: (C)

The following paragraphs illustrate one example exemplar prompt obtained from the PubMedQA datasets, using the chain-of-thought prompt structure:

Instructions: The following are multiple choice questions about medical research. Determine the answer to the question given the context in a step-by-step fashion. Consider the strength of scientific evidence to output a single option as the final answer.

Context: To describe the interstitial fluid (ISF) and plasma pharmacokinetics of meropenem in patients on continuous venovenous haemodiafiltration (CVVHDF). This was a prospective observational pharmacokinetic study. Meropenem (500 mg) was administered every 8 h. CVVHDF was targeted as a 2-3 L/h exchange using a polyacrylonitrile filter with a surface area of 1.05 m2 and a blood flow rate of 200 mL/min. Serial blood (pre- and post-filter), filtrate/dialysate and ISF concentrations were measured on 2 days of treatment (Profiles A and B). Subcutaneous tissue ISF concentrations were determined using microdialysis. A total of 384 samples were collected. During Profile A, the comparative median (IQR) ISF and plasma peak concentrations were 13.6 (12.0-16.8) and 40.7 (36.6-45.6) mg/L and the trough concentrations were 2.6 (2.4-3.4) and 4.9 (3.5-5.0) mg/L, respectively. During Profile B, the ISF trough concentrations increased by ˜40%. Meropenem ISF penetration was estimated at 63% (60%-69%) and 69% (65%-74%) for Profiles A and B, respectively, using comparative plasma and ISF AUCs. For Profile A, the plasma elimination t½ was 3.7 (3.3-4.0) h, the volume of distribution was 0.35 (0.25-0.46) L/kg, the total clearance was 4.1 (4.1-4.8) L/h and the CVVHDF clearance was 2.9 (2.7-3.1) L/h.

Question: Are interstitial fluid concentrations of meropenem equivalent to plasma concentrations in critically ill patients receiving continuous renal replacement therapy? (A) Yes (B) No (C) Maybe

Explanation: This is the first known report of concurrent plasma and ISF concentrations of a meropenem antibiotic during CVVHDF. We observed that the ISF concentrations of meropenem were significantly lower than the plasma concentrations, although the present dose was appropriate for infections caused by intermediately susceptible pathogens (MIC≤4 mg/L).

Answer: (B)

Context: Family caregivers of dementia patients are at increased risk of developing depression or anxiety. A multi-component program designed to mobilize support of family networks demonstrated effectiveness in decreasing depressive symptoms in caregivers. However, the impact of an intervention consisting solely of family meetings on depression and anxiety has not yet been evaluated. This study examines the preventive effects of family meetings for primary caregivers of community-dwelling dementia patients. A randomized multicenter trial was conducted among 192 primary caregivers of community dwelling dementia patients. Caregivers did not meet the diagnostic criteria for depressive or anxiety disorder at baseline. Participants were randomized to the family meetings intervention (n=96) or usual care (n=96) condition. The intervention consisted of two individual sessions and four family meetings which occurred once every 2 to 3 months for a year. Outcome measures after 12 months were the incidence of a clinical depressive or anxiety disorder and change in depressive and anxiety symptoms (primary outcomes), caregiver burden and quality of life (secondary outcomes). Intention-to-treat as well as per protocol analyses were performed. A substantial number of caregivers (72/192) developed a depressive or anxiety disorder within 12 months. The intervention was not superior to usual care either in reducing the risk of disorder onset (adjusted IRR 0.98; 95% CI 0.69 to 1.38) or in reducing depressive (randomization-by-time interaction coefficient=−1.40; 95% CI-3.91 to 1.10) or anxiety symptoms (randomization-by-time interaction coefficient=−0.55; 95% CI-1.59 to 0.49). The intervention did not reduce caregiver burden or their health related quality of life.

Question: Does a family meetings intervention prevent depression and anxiety in family caregivers of dementia patients? (A) Yes (B) No (C) Maybe

Explanation: This study did not demonstrate preventive effects of family meetings on the mental health of family caregivers. Further research should determine whether this intervention might be more beneficial if provided in a more concentrated dose, when applied for therapeutic purposes or targeted towards subgroups of caregivers.

Answer: (B)

Context: To compare adherence to follow-up recommendations for colposcopy or repeated Papanicolaou (Pap) smears for women with previously abnormal Pap smear results. Retrospective cohort study. Three northern California family planning clinics. All women with abnormal Pap smear results referred for initial colposcopy and a random sample of those referred for repeated Pap smear. Medical records were located and reviewed for 90 of 107 women referred for colposcopy and 153 of 225 women referred for repeated Pap smears. Routine clinic protocols for follow-up-telephone call, letter, or certified letter-were applied without regard to the type of abnormality seen on a Pap smear or recommended examination. Documented adherence to follow-up within 8 months of an abnormal result. Attempts to contact the patients for follow-up, adherence to follow-up recommendations, and patient characteristics were abstracted from medical records. The probability of adherence to follow-up vs the number of follow-up attempts was modeled with survival analysis. Cox proportional hazards models were used to examine multivariate relationships related to adherence. The rate of overall adherence to follow-up recommendations was 56.0% (136/243). Adherence to a second colposcopy was not significantly different from that to a repeated Pap smear (odds ratio, 1.40; 95% confidence interval, 0.80-2.46). The use of as many as 3 patient reminders substantially improved adherence to follow-up. Women without insurance and women attending 1 of the 3 clinics were less likely to adhere to any follow-up recommendation (hazard ratio for no insurance, 0.43 [95% confidence interval, 0.20-0.93], and for clinic, 0.35 [95% confidence interval, 0.15-0.73]).

Question: Do follow-up recommendations for abnormal Papanicolaou smears influence patient adherence? (A) Yes (B) No (C) Maybe

Explanation: Adherence to follow-up was low in this family planning clinic population, no matter what type of follow-up was advised. Adherence was improved by the use of up to 3 reminders. Allocating resources to effective methods for improving adherence to follow-up of abnormal results may be more important than which follow-up procedure is recommended.

Answer: (B)

The following paragraphs illustrate one example exemplar prompt obtained from the MMLU datasets, using the chain-of-thought prompt structure:

Instructions: The following are multiple-choice questions about medical knowledge. Solve them in a step-by-step fashion. Output a single option as the final answer.

Question: The energy for all forms of muscle contraction is provided by: (A) ATP. (B) ADP. (C) phosphocreatine. (D) oxidative phosphorylation.

Explanation: The sole fuel for muscle contraction is adenosine triphosphate (ATP). During near maximal intense exercise the muscle store of ATP will be depleted in less than one second. Therefore, to maintain normal contractile function ATP must be continually resynthesized. These pathways include phosphocreatine and muscle glycogen breakdown, thus enabling substrate-level phosphorylation (‘anaerobic’) and oxidative phosphorylation by using reducing equivalents from carbohydrate and fat metabolism (‘aerobic’).

Answer: (A)

Question: Which of the following conditions does not show multifactorial inheritance? (A) Pyloric stenosis (B) Schizophrenia (C) Spina bifida (neural tube defects) (D) Marfan syndrome

Explanation: Multifactorial inheritance refers to when a condition is caused by multiple factors, which may be both genetic or environmental. Marfan is an autosomal dominant trait. It is caused by mutations in the FBNI gene, which encodes a protein called fibrillin-1. Hence, Marfan syndrome is not an example of multifactorial inheritance.

Answer: (D)

Question: What is the embryological origin of the hyoid bone? (A) The first pharyngeal arch (B) The first and second pharyngeal arches (C) The second pharyngeal arch (D) The second and third pharyngeal arches

Explanation: In embryology, the pharyngeal arches give rise to anatomical structure in the head and neck. The hyoid bone, a small bone in the midline of the neck anteriorly, is derived from the second and third pharyngeal arches.

Answer: (D)

Question: In a given population, 1 out of every 400 people has a cancer caused by a completely recessive allele, b. Assuming the population is in Hardy-Weinberg equilibrium, which of the following is the expected proportion of individuals who carry the b allele but are not expected to develop the cancer? (A) 1/400 (B) 19/400 (C) 20/400 (D) 38/400

Explanation: The expected proportion of individuals who carry the b allele but are not expected to develop the cancer equals to the frequency of heterozygous allele in the given population. According to the Hardy-Weinberg equation p\hat{ }2+2pq+q\hat{ }2=1, where p is the frequency of dominant allele frequency, q is the frequency of recessive allele frequency, p\hat{ }2 is the frequency of the homozygous dominant allele, q\hat{ }2 is the frequency of the recessive allele, and 2pq is the frequency of the heterozygous allele. Given that q\hat{ }2=1/400, hence, q=0.05 and p=1−q=0.95. The frequency of the heterozygous allele is 2pq=2*0.05*0.95=38/400.

Answer: (D)

Question: A high school science teacher fills a 1 liter bottle with pure nitrogen and seals the lid. The pressure is 1.70 atm, and the room temperature is 25° C. Which two variables will both increase the pressure of the system, if all other variables are held constant? (A) Decreasing volume, decreasing temperature (B) Increasing temperature, increasing volume (C) Increasing temperature, increasing moles of gas (D) Decreasing moles of gas, increasing volume

Explanation: According to the ideal gas law, PV=nRT (P=pressure, V=volume, n=number of moles, R=gas constant, T=temperature). Hence, increasing both temperature (T) and moles of gas (n), while other variables stay constant, will indeed increase the pressure of the system.

Answer: (C)

Question: A 22-year-old male marathon runner presents to the office with the complaint of right-sided rib pain when he runs long distances. Physical examination reveals normal heart and lung findings and an exhalation dysfunction at ribs 4-5 on the right. Which of the following muscles or muscle groups will be most useful in correcting this dysfunction utilizing a direct method? (A) anterior scalene (B) latissimus dorsi (C) pectoralis minor (D) quadratus lumborum

Explanation: All of the muscles have an insertion on the rib cage; however only one has an insertion at ribs 4-5 and could be responsible for right-sided rib pain: pectoralis minor. Pectoralis minor inserts to the costal cartilage of the anterior third to fifth ribs.

Answer: (C)

Other data can be used in addition or alternative to medical question-answer data. Electronic medical records (EMR) can be used to provide or learn prompt data, assuming the requisite permissions are granted. EMR can include notes (e.g., practitioner notes from patient visits, etc.), lab test results or other tests or diagnostic procedures, prescription information or other courses of pharmacological treatment, etc. EMR can include image data (e.g., data associated with X-rays, CT scans, MRI, Ultrasound, PET scans, SPECT scans, Mammograms, bone scans, fluoroscopy, angiography, endoscopy, colonoscopy, echocardiogram, electroencephalogram (EEG), positron emission mammography (PEM) optical coherence tomography (OCT), thermography, nuclear medicine imaging, digital radiography, computed radiography, etc.). EMR can include sensor data (e.g., data associated with blood glucose sensors, electrocardiogram sensors, blood oxygen sensors, blood pressure sensors, temperature sensors, electroencephalogram sensors, respiratory rate sensors, pulse sensors, magnetic resonance imaging (MRI) sensors, and electrooculography (EOG) sensors, etc.). EMR can include data from wearable devices, such as smartwatches and the like.

Example EMR data can be input as an exemplar prompt along with an expert's natural language description of a task performed with respect to the EMR. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the EMR, a health-related/diagnostic search or summary task based on the EMR, an administrative or record-related question answering task based on the EMR, an administrative or record-related search or summary task based on the EMR, a patient clustering or comparison task based on the EMR, a note-taking or augmentation task based on the EMR, an updating task to update the EMR based on the current EMR, a billing or insurance claim task based on the EMR, a clinical trial evaluation task based on the EMR (e.g., investigation of side effects), etc.

Clinical trials data can be used to provide or learn prompt data, assuming the requisite permissions are granted. Clinical trials data can include notes (e.g., practitioner notes from patient visits, etc.), lab test results or other tests or diagnostic procedures, prescription information or other courses of pharmacological treatment, etc. Clinical trials data can include medical history data for participants' past and current medical conditions, medications, and allergies. Clinical trials data can include baseline measurements taken before participants start taking the study drug. Clinical trials data can include treatment data that includes details about the study drug, such as the dose, frequency, and duration of treatment. Clinical trials data can include outcome data that includes how participants respond to the study drug, such as changes in their symptoms, quality of life, or overall health. Clinical trials data can include adverse event data that are any negative side effects that participants experience while taking the study drug. Clinical trials data can include other factors, such as participants' lifestyle, diet, and environment.

Example clinical trials data can be input as an exemplar prompt along with an expert's natural language description of a task performed with respect to the clinical trials data. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the clinical trials data, a health-related/diagnostic search or summary task based on the clinical trials data, an administrative or record-related question answering task based on the clinical trials data, an administrative or record-related search or summary task based on the clinical trials data, a patient clustering or comparison task based on the clinical trials data, a note-taking or augmentation task based on the clinical trials data, an updating task to update the clinical trials data based on the current clinical trials data, a billing or insurance claim task based on the clinical trials data, a clinical trial evaluation task based on the clinical trials data (e.g., investigation of side effects), etc.

Medical and science literature data can be used to provide or learn prompt data, assuming the requisite permissions are granted. Medical and science literature data can include the contents (e.g., text, images, multimedia, raw data, etc.) of research articles, textbooks, public guidelines, drug package inserts, clinical trial reports, etc. Example medical and science literature data can be input as an exemplar prompt along with an expert's natural language description of a task performed with respect to the medical and science literature data. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the medical and science literature data, a health-related/diagnostic search or summary task based on the medical and science literature data, an administrative or record-related question answering task based on the medical and science literature data, an administrative or record-related search or summary task based on the medical and science literature data, a patient clustering or comparison task based on the medical and science literature data, a note-taking or augmentation task based on the medical and science literature data, an updating task to update the medical and science literature data based on the current medical and science literature data, a billing or insurance claim task based on the medical and science literature data, a clinical trial evaluation task based on the medical and science literature data (e.g., investigation of side effects), etc.

Patient forum data can be used to provide or learn prompt data, assuming the requisite permissions are granted. Patient forum data can include the contents (e.g., text, images, multimedia, etc.) of online discussion boards or other support group discussion threads. Example patient forum data a can be input as an exemplar prompt along with an expert's natural language description of a task performed with respect to the patient forum data. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the patient forum data, a health-related/diagnostic search or summary task based on the patient forum data, an administrative or record-related question answering task based on the patient forum data, an administrative or record-related search or summary task based on the patient forum data, a patient clustering or comparison task based on the patient forum data, a note-taking or augmentation task based on the patient forum data, an updating task to update the patient forum data based on the current patient forum data, a billing or insurance claim task based on the patient forum data, a clinical trial evaluation task based on the patient forum data (e.g., investigation of side effects), etc.

Patent data can be used to provide or learn prompt data. Patent data can include the contents (e.g., text, figures, etc.) of patent publications or statistics descriptive thereof (e.g., filing statistics, assignment records, maintenance status, etc.). Example patent data a can be input as an exemplar prompt along with an expert's natural language description of a task performed with respect to the patent data. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the patent data, a health-related/diagnostic search or summary task based on the patent data, an administrative or record-related question answering task based on the patent data, an administrative or record-related search or summary task based on the patent data, a patient clustering or comparison task based on the patent data, a note-taking or augmentation task based on the patent data, an updating task to update the patent data based on the current patent data, a billing or insurance claim task based on the patent data, a clinical trial evaluation task based on the patent data (e.g., investigation of side effects), a whitespace or freedom to operate analysis based on the patent data, etc.

Omics data can be used to provide or learn prompt data. Omics data can include Genomics data. Genomics can include the study of the genome, which is the complete set of DNA in an organism. Genomics data can be used to identify genes, understand gene function, and track changes in the genome over time. Omics data can include Transcriptomics data. Transcriptomics can include the study of the transcriptome, which is the complete set of RNA transcripts from DNA. Transcriptomics data can be used to identify genes that are being expressed, understand how genes are regulated, and track changes in gene expression over time. Omics data can include Proteomics data. Proteomics can include the study of the proteome, which is the complete set of proteins expressed by an organism. Proteomics data can be used to identify proteins, understand protein function, and track changes in protein expression over time. Omics data can include Metabolomics data. Metabolomics can include the study of the metabolome, which is the complete set of small molecules in an organism. Metabolomics data can be used to identify metabolites, understand metabolic pathways, and track changes in metabolism over time.

Example omics data a can be input as an exemplar prompt along with an expert's natural language description of a task performed with respect to the omics data. Generally, the tasks can include question answering tasks, summarization tasks, translation tasks (e.g., translate language, translate perspective or voice, such as patient to doctor, expert to non-expert, etc.), prediction tasks, classification tasks, and the like. For example, the task can be a health related/diagnostic question answering task based on the omics data, a health-related/diagnostic search or summary task based on the omics data, an administrative or record-related question answering task based on the omics data, an administrative or record-related search or summary task based on the omics data, a patient clustering or comparison task based on the omics data, a note-taking or augmentation task based on the omics data, an updating task to update the omics data based on the current omics data, a billing or insurance claim task based on the omics data, a clinical trial evaluation task based on the omics data (e.g., investigation of side effects), a drug discovery or simulation task based on the omics data, etc.

### Example Applications

The techniques of the present disclosure can be used to perform a variety of tasks in specialized knowledge domains. The techniques of the present disclosure can be used to perform a variety of tasks in a medical or clinical domain. For instance, the instruction prompt tuning techniques of the present disclosure can align machine-learned models to specialized domains in a data-efficient manner.

Further, fine-tuning techniques can be applied in lieu of or in addition to instruction prompt tuning. For example, for the example applications noted below, the model(s) can also be fine-tuned on the corresponding datatypes for alignment in those domains.

For example, a user can query machine-learned model 108 for obtaining information for learning medicine. For instance, a student-facing software tool can provide an input interface to input queries to be used in an input sequence 104 to be processed by machine-learned model 108. For instance, input queries can seek knowledge recall (e.g., “Please list bones in the body.”), knowledge exposition (e.g., “Please explain the muscles activated during a sneeze.”), knowledge testing (e.g., “Please write a quiz testing anatomy at the level of a first-year medical student.”), imaging (e.g., “Please generate an image showing the appearance of a healthy esophageal wall.”), audio (e.g., “What does an arrythmia sound like?”), note-taking (e.g., “Please listen to the audio of [class] and take detailed notes prefaced by a brief summary”), and the like. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for medical education, exemplar prompt data can include pedagogical exemplars. For instance, exemplar prompt data can include example explanations of complex topics written by professors addressed to student audiences, example test questions curated by professors for students, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include professors, students, etc. In this manner, for instance, the exemplar prompt data can help align machine-learned model 108 to a medical education domain.

For example, a user can query machine-learned model 108 for obtaining information by searching medical research. For instance, a student-facing software tool can provide an input interface to input queries to be used in an input sequence 104 to be processed by machine-learned model 108. For instance, input queries can be directed to search medical research documents to summarize a number of reports or articles on a particular topic. Machine-learned model 108 can evaluate a number of research articles and determine a consensus, lack of consensus, majority opinion, plurality opinion, strong minority opinion, etc. Machine-learned model 108 can evaluate white space in topic coverage. For instance, a student searching for a new thesis topic can query machine-learned model 108 to identify sparsely researched areas, such as areas of new interest. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for querying medical research from a student perspective, exemplar prompt data can include exemplars of evaluations of medical research written to a student audience. For instance, exemplar prompt data can include example explanations of complex topics written by professors or researchers but addressed to student audiences, summarizations of research reports using terminology familiar to a student population, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include researchers, students, etc. In this manner, for instance, the exemplar prompt data can help align machine-learned model 108 to perform queries over medical research documents.

For example, a professional user can query machine-learned model 108 for obtaining information by searching medical research. For instance, a clinical researcher-facing software tool can provide an input interface to input queries to be used in an input sequence 104 to be processed by machine-learned model 108. For instance, input queries can be directed to search medical research documents to summarize a number of reports or articles on a particular topic. Machine-learned model 108 can evaluate a number of research articles and determine a consensus, lack of consensus, majority opinion, plurality opinion, strong minority opinion, etc. Machine-learned model 108 can evaluate a number of research articles and summarize a history of use of a particular pharmacological or other medical intervention. Machine-learned model 108 can evaluate risk and annotate potential adverse effects of new drug formulations based on review of medical literature. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for querying medical research from a research perspective, exemplar prompt data can include exemplars of evaluations of medical research written to a research audience (e.g., a default style for many research papers). For instance, exemplar prompt data can include example explanations of complex topics written by researchers for researchers, summarizations of research reports using terminology familiar to a researcher population, isolation of important key results from a discussion section of a paper, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include researchers, medical professionals, etc. In this manner, for instance, the exemplar prompt data can help align machine-learned model 108 to perform queries over medical research documents.

For example, a user can query machine-learned model 108 to generate summarization of discharge notes (e.g., discharge from a hospital stay). For instance, a software tool can provide an input interface to input queries to be used in an input sequence 104 to be processed by machine-learned model 108. For instance, input queries can be directed to generating a summary of discharge notes. During a hospital stay or other inpatient interval, numerous health records can be generated to describe the patient's experience, health, and treatment plan going forward. Such health records can be extensive and laborious to process. Machine-learned model 108 can process the patient's health records and generate a summary for discharge notes. The machine-learned model 108 can be instructed to write the discharge notes in a style configured to improve interpretability by a patient. The machine-learned model 108 can be instructed to write the discharge notes in a style configured to improve speed and precision of reading by a medical professional. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for generating discharge notes and summaries thereof, exemplar prompt data can include exemplars of discharge notes and the records from which the notes are derived. For instance, exemplar prompt data can include example health records (real records, synthetic records, etc.) following a particular course of treatment for a patient. The exemplar prompt data can include instructions indicating the target audience (e.g., patient, medical professional, etc.). The exemplar prompt data can include example discharge notes written by a medical professional. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, patients, patient caregivers, etc. In this manner, for instance, the exemplar prompt data can help align machine-learned model 108 to generate discharge notes and summaries thereof.

For example, a user can query machine-learned model 108 to generate summarization of clinician notes (e.g., resulting from a clinical interaction with a patient, in a lab, etc.). For instance, a software tool can provide an input interface to input queries to be used in an input sequence 104 to be processed by machine-learned model 108. For instance, input queries can be directed to generating a summary of clinician notes. Clinical tasks can be very complex and require extensive note keeping to maintain rigorous standards of repeatability, accuracy, auditability, etc. Machine-learned model 108 can process a set of clinical notes and generate a summary for quick reference or publication. The machine-learned model 108 can be instructed to write the clinical notes in a style configured to improve interpretability in a research publication, regulatory report, internal clinical memorandum, etc. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for generating clinical notes and summaries thereof, exemplar prompt data can include exemplars of clinical notes and the raw experiment data or structure that was the subject of the note keeping. For instance, exemplar prompt data can include example health records (real records, synthetic records, etc.) following a particular course of treatment for a patient. Exemplar prompt data can include example lab records, sensor outputs, experimental results, etc. The exemplar prompt data can include instructions indicating the target audience (e.g., patient, medical professional, researcher, regulator, etc.). The exemplar prompt data can include example clinical notes written by a clinician. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, patients, researchers regulators, etc. In this manner, for instance, the exemplar prompt data can help align machine-learned model 108 to generate clinical notes and summaries thereof.

For example, a user can query machine-learned model 108 to explain a disease condition to a patient or other non-expert. The user can be the patient or a medical personnel attending to the patient or other caregiver. For instance, a software tool can provide an input interface to input queries to be used in an input sequence 104 to be processed by machine-learned model 108. For instance, input queries can be directed to generating an explanation of a disease condition in a manner understandable by a patient. Disease conditions can be complex and have many possible courses of progression. Explaining disease conditions to a patient in a manner that is accurate and educational, while maintaining sensitivity to the emotional response to hearing the information can be a challenging task. Machine-learned model 108 can process input information about the patient and about the disease and return an output that explains the disease to the intended audience. Machine-learned model 108 can output content delivered in one or multiple modalities: audio content for the patient to hear; video content for the patient to watch; visual aids, such as slide decks; textual content for the patient to read, etc.

To align machine-learned model 108 for explaining disease conditions, exemplar prompt data can include exemplars of disease conditions, patient characteristics, and how a person explained the disease to the patient. For instance, exemplar prompt data can include example health records (real records, synthetic records, etc.) following a particular course of treatment for a patient. Exemplar prompt data can include data describing the patient's response during the treatment (e.g., physiological response, emotional response, verbal response, etc.). Exemplar prompt data can include instructions indicating the target audience (e.g., patient, caregiver, etc.). Exemplar prompt data can include example explanations of a disease condition. The example explanations can include audio content for the patient to hear; video content for the patient to watch; visual aids, such as slide decks; textual content for the patient to read, etc.

Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, patients, caregivers, psychiatrists, therapists, etc. In this manner, for instance, the exemplar prompt data can help align machine-learned model 108 to explain disease conditions.

More generally, a user can query machine-learned model 108 to assist with physician communications, such as physician-patient communications. The user can be the patient or a medical personnel attending to the patient or other caregiver. For instance, a software tool can provide an input interface to input queries to be used in an input sequence 104 to be processed by machine-learned model 108. For instance, input queries can be directed to translating a desired communication from a style familiar to physicians to a style more understandable by non-physicians. Physician communications can often contain complex medical jargon, often without sufficient context to derive the meaning of such terms. This can lead to difficulties communicating between physicians and non-physicians. Machine-learned model 108 can process input information about the audience (e.g., age, competency, level of education, etc.) and about the desired message to be communicated and return an output for delivering the message to the intended audience. Machine-learned model 108 can output content delivered in one or multiple modalities: audio content for the audience to hear; video content for the audience to watch; visual aids, such as slide decks; textual content for the audience to read (or to be read to the audience), etc.

To align machine-learned model 108 for assisting physician communications, exemplar prompt data can include exemplars of physician messages, audience characteristics, and how a person delivered an appropriate communication to the audience. For instance, exemplar prompt data can include example health records (real records, synthetic records, etc.) following a particular course of treatment for a patient. Exemplar prompt data can include data describing the patient's response during the treatment (e.g., physiological response, emotional response, verbal response, etc.). Exemplar prompt data can include instructions indicating the target audience (e.g., patient, caregiver, etc.). Exemplar prompt data can include example messages that were delivered with sensitivity to the audience context. The example explanations can include audio content for the audience to hear; video content for the audience to watch; visual aids, such as slide decks; textual content for the audience to read (or to be read to the audience), etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, patients, caregivers, psychiatrists, therapists, etc. In this manner, for instance, the exemplar prompt data can help align machine-learned model 108 to assist physician communications.

For example, a user can query machine-learned model 108 to query a repository of adverse events (e.g., related to a clinical trial or otherwise occurring in a patient population). The user can be a medical personnel. For instance, a software tool can provide an input interface to input queries to be used in an input sequence 104 to be processed by machine-learned model 108. For instance, input queries can be directed to identifying meaningful trends or associations between adverse events and other factors (e.g., treatments, drugs, hospital conditions, or other interactions or interventions). Adverse event data and data describing all external factors possibly associated therewith can be expansive and difficult if not impossible to parse manually. Some example factors include the severity of the disease or condition being studied, the type of treatment being studied, the dose of the treatment being studied, the length of time that participants are taking the treatment, the age and health of the participants, the participants' lifestyle habits (e.g., smoking, alcohol consumption, exercise, diet, etc.), the participants' genetic makeup, etc. Machine-learned model 108 can process input information about the adverse events and other factors to provide relevant outputs. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for querying adverse events, exemplar prompt data can include exemplars of adverse event information and a set of environmental or other external factors that were associated with the adverse event. For instance, exemplar prompt data can include example health records (real records, synthetic records, etc.) following a particular course of treatment for a patient. Exemplar prompt data can include data describing the patient's response during the treatment (e.g., physiological response, emotional response, verbal response, etc.). Exemplar prompt data can include instructions indicating the target audience (e.g., medical professional, clinician, scientist, regulator, etc.). Exemplar prompt data can include example associations between adverse events and other factors, such as: the severity of the disease or condition being studied, the type of treatment being studied, the dose of the treatment being studied, the length of time that participants are taking the treatment, the age and health of the participants, the participants' lifestyle habits (e.g., smoking, alcohol consumption, exercise, diet, etc.), the participants' genetic makeup, etc. Exemplar prompt data can include examples of factors that were not associated with adverse events. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, patients, caregivers, psychiatrists, therapists, etc. In this manner, for instance, the exemplar prompt data can help align machine-learned model 108 to query adverse events.

Similarly, for example, a user can query machine-learned model 108 to query a lab notebook (e.g., related to a clinical trial or other medical enterprise). The user can be a medical personnel. For instance, a software tool can provide an input interface to input queries to be used in an input sequence 104 to be processed by machine-learned model 108. For instance, input queries can be directed to analyzing or summarizing material from the lab notebook, such as to identify meaningful trends or associations between events and external factors (e.g., treatments, drugs, hospital conditions, or other interactions or interventions). Lab notebooks can be used to document the research and development of new drugs and treatments, and thus contain substantial amounts of critical data. They can also be used to document the clinical trials of new drugs and treatments. Machine-learned model 108 can process input information to generate outputs about the lab notebook contents (e.g., summaries and analysis thereof). Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for querying adverse events, exemplar prompt data can include exemplars of adverse event information and a set of environmental or other external factors that were associated with the adverse event. For instance, exemplar prompt data can include example health records (real records, synthetic records, etc.) following a particular course of treatment for a patient. Exemplar prompt data can include data describing the patient's response during the treatment (e.g., physiological response, emotional response, verbal response, etc.). Exemplar prompt data can include instructions indicating the target audience (e.g., medical professional, clinician, scientist, regulator, etc.). Exemplar prompt data can include experimental descriptions and results. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, scientists, research assistants, clinicians, regulators, etc. In this manner, for instance, the exemplar prompt data can help align machine-learned model 108 to query lab notebooks.

For example, a user can query machine-learned model 108 to assist with care navigation (e.g., in hospitals). Care navigation can be helpful for patients with a variety of needs, including those who are newly diagnosed with a chronic condition, those who are struggling to manage their care, and those who are facing financial barriers to care. Care navigation can help patients improve their health outcomes, reduce their costs, and improve their overall satisfaction with their care. The user can be a medical personnel or a patient. For instance, a software tool can provide an input interface to input queries to be used in an input sequence 104 to be processed by machine-learned model 108. For instance, input queries can be directed to understanding how to manage a care plan, how to obtain financial assistance for care, etc. Machine-learned model 108 can process input information to generate outputs about care navigation. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for care navigation, exemplar prompt data can include exemplars of patient care plans, available resources, and guidance on how to navigate the care plan in view of the available resources. For instance, exemplar prompt data can include example health records (real records, synthetic records, etc.) following a particular course of treatment for a patient. Exemplar prompt data can include data describing the patient's response during the treatment (e.g., physiological response, emotional response, verbal response, etc.). Exemplar prompt data can include instructions indicating the target audience (e.g., patient, caregiver, etc.). Exemplar prompt data can include care navigation advice from experts, such as from medical professionals, care advisors or advocates, experienced caregivers, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, patients, caregivers, patient rights advisors and advocates, etc. In this manner, for instance, the exemplar prompt data can help align machine-learned model 108 to assist with care navigation.

For example, a user can query machine-learned model 108 to assist with insurance billing or other billing tasks. A billing code match is the process of ensuring that the codes used to bill for healthcare services are accurate and match the services that were actually provided. This helps to ensure that patients or their insurers are billed correctly and that insurance companies are paying the correct amount. This can include reviewing the healthcare documentation to identify the services that were provided and selecting the appropriate billing codes for the services that were provided. The user can be a medical personnel, a patient, and insurance personnel, a legal representative, etc. For instance, a software tool can provide an input interface to input queries to be used in an input sequence 104 to be processed by machine-learned model 108. For instance, input queries can be directed to identifying appropriate billing codes that match the plan of care provided to a patient (e.g., retrospectively or prospectively). Machine-learned model 108 can process input information about a treatment to generate outputs indicating billing information. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for billing tasks, exemplar prompt data can include exemplars of electronic health record snippets (assuming requisite permissions are granted) and associated billing data (e.g., corresponding billing codes). For instance, exemplar prompt data can include example health records (real records, synthetic records, etc.) following a particular course of treatment for a patient. Exemplar prompt data can include summaries of example health records (real records, synthetic records, etc.) following a particular course of treatment for a patient. Exemplar prompt data can include instructions indicating the target audience (e.g., patient, insurer, etc.). Exemplar prompt data can include billing data vetted and confirmed by experts, e.g., insurance experts, legal experts, medical professionals, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, patients, caregivers, patient rights advisors and advocates, insurance representatives, etc. In this manner, for instance, the exemplar prompt data can help align machine-learned model 108 to assist with billing tasks.

For example, a user can query machine-learned model 108 to assist with clinical protocol authoring and updating. Machine-learned model 108 can assist with writing a clinical protocol in a clear and concise manner that is understandable to experts and non-experts. It can be comprehensive enough to allow others to replicate the study. It can be concise so that it is not unduly difficult to read and understand. Machine-learned model 108 can draft a protocol to include various sections. Machine-learned model 108 can draft an introduction that provides an overview of the study, including the research question, the study population, and the study objectives. Machine-learned model 108 can draft background material to provide a brief overview of the scientific background of the study, including the relevant literature and the rationale for the study. Machine-learned model 108 can draft a methods section to describe the study design, the study population, the interventions, the outcome measures, and the data collection and analysis methods. Machine-learned model 108 can draft a statistical analysis plan to describe the statistical methods that will be used to analyze the data. Machine-learned model 108 can draft discussion of the implications of the study, including the limitations of the study and the need for further research. Machine-learned model 108 can update the protocol as needed throughout the course of the study. Updates can be triggered automatically (e.g., periodically, based on a triggering event, etc.) or manually. The user can be a medical personnel, a clinician, a researcher, etc. For instance, a software tool can provide an input interface to input queries to be used in an input sequence 104 to be processed by machine-learned model 108. For instance, input queries can be directed to authoring a clinical trial protocol or portions thereof. Machine-learned model 108 can process input information about the subject(s) of the trial to output a protocol for studying the subjects. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for drafting clinical protocol materials, exemplar prompt data can include exemplars of study subjects (e.g., an effect of a drug or other medical interventions) and study protocols used to evaluate the study subjects. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, clinical trial participants, etc. In this manner, for instance, the exemplar prompt data can help align machine-learned model 108 to assist with clinical protocol drafting tasks.

For example, a user can query machine-learned model 108 to assist with clinical trial matching. Clinical trial matching services can make it easier for patients to find clinical trials that are a good fit for them. Clinical trial matching services can take into account a patient's individual needs and circumstances when matching them with clinical trials. Machine-learned model 108 can process input data describing a set of available clinical trials and input data describing a patient's health or healthcare needs. The user can be a medical personnel, a clinician, a researcher, a patient, etc. For instance, a software tool can provide an input interface to input queries to be used in an input sequence 104 to be processed by machine-learned model 108. For instance, input queries can be directed to matching a patient to a clinical trial out of a set of clinical trials. Input queries can be directed to matching a clinical trial to a patient out of a set of patients. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for clinical trial matching, exemplar prompt data can include exemplars of matching a patient to a clinical trial out of a set of clinical trials, matching a clinical trial to a patient out of a set of patients, or both. Exemplar prompt data can include the basis for the matching decision, including health record data, clinical trial descriptions or protocols, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, clinical trial participants, etc. In this manner, for instance, the exemplar prompt data can help align machine-learned model 108 to assist with clinical matching tasks.

For example, a user can query machine-learned model 108 to assist with patient intake. For instance, machine-learned model 108 can operate as a patient intake chatbot for conversing with a patient or caregiver for initiating intake of the patient to a care facility, clinical trial, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the chatbot. Machine-learned model 108 can process input information about the patient and a set of intake procedures to output, to the patient, another part of a dialogue as the chatbot. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, the intake data for intaking the patient to the care facility or clinical trial. Machine-learned model 108 can flag any errors or omissions in the intake data. Machine-learned model 108 can flag any health concerns that machine-learned model 108 identifies in the intake data.

To align machine-learned model 108 for operating as a patient intake chatbot, exemplar prompt data can include exemplars of conversational interchanges between patient and healthcare provider regarding intake (e.g., following an intake procedure). Exemplar prompt data can include potential questions asked by the healthcare provider or the patient and appropriate or anticipated responses thereto, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with patient intake tasks.

For example, a user can query machine-learned model 108 to provide behavioral nudges. Nudges can be used to increase patient health in a number of ways, including improving medication adherence. Nudges can be used to help patients remember to take their medications, such as by sending them text reminders or providing them with pill boxes with built-in alarms. Nudges can be used for encouraging healthy behaviors. Nudges can be used to encourage patients to make healthier choices, such as by providing them with healthy food options in hospitals or by making it easier for them to exercise. Nudges can be used for reducing healthcare costs. Nudges can be used to reduce healthcare costs by encouraging patients to make more efficient use of resources, such as by scheduling appointments online or by using telehealth services. Machine-learned model 108 can operate to send communications or alerts or other interventions to a device associated with a subject of the nudge (e.g., a patient). The user can be a medical personnel, a clinician, a researcher, a patient, a caregiver, etc. who desires to initiate a nudging plan. The user can input a goal or desired schedule or target for the nudging plan, and machine-learned model 108 can generate nudges based on the inputs. The nudges can be generated for the user to then implement (e.g., to perform actions or to schedule reminders or calendar events or haptic alerts according to a schedule or protocol output by machine-learned model 108). The nudges can be automatically implemented by machine-learned model 108. Machine-learned model 108 can interface with one or more tools to send communications (e.g., emails, text messages), schedule alerts, or otherwise interact with the subject of the nudge plan, such as by activating actions or outputs on wearable devices. For instance, a software tool can provide an input interface to input queries to be used in an input sequence 104 to be processed by machine-learned model 108. For instance, input queries can form requests for nudges or a plan or protocol for conducting nudges. Machine-learned model 108 can process input information about the patient and a set of intake procedures to output nudges or a protocol or plan for nudging. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 to provide behavioral nudges, exemplar prompt data can include exemplars of patient health conditions and patient health data, for one or multiple patients (e.g., population-level data), patient health goals, and nudges that are effective to guide the patient toward the health goals. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, psychiatrists, behavioral scientists, legal advocates, patients, caregivers, clinical trial participants, etc. In this manner, for instance, the exemplar prompt data can help align machine-learned model 108 to assist with nudging tasks.

For example, a user can query machine-learned model 108 to assist with remote check-ins. For instance, machine-learned model 108 can operate as a patient check-in chatbot for conversing with a patient or caregiver for initiating a follow up or remote check-in with the patient or caregiver after a visit to a care facility, clinical trial, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can initiate or form part of a dialogue with the chatbot. Machine-learned model 108 can process input information about the patient and a set of check-in procedures to output, to the patient, part of a dialogue as the chatbot. For instance, a medical personnel can provide an initial input to initialize a conversation between machine-learned model 108 and a patient. The initial input can provide a current status of care and a set of desired information to obtain from the check-in interaction. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, the check-in data for updating patient records after a visit to the care facility or clinical trial. Machine-learned model 108 can flag any errors or omissions in the check-in data. Machine-learned model 108 can flag any health concerns that machine-learned model 108 identifies in the check-in data.

To align machine-learned model 108 for operating as a patient check-in chatbot, exemplar prompt data can include exemplars of conversational interchanges between patient and healthcare provider regarding check-ins (e.g., following an intervention). Exemplar prompt data can include potential questions asked by the healthcare provider or the patient and appropriate or anticipated responses thereto, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with patient check-in tasks.

For example, a user can query machine-learned model 108 to assist with patient onboarding for clinical trials. Steps involved in patient onboarding for clinical trials can include patient recruitment to recruit patients who are eligible to participate in the clinical trial. This can be done through a variety of methods, such as advertising, patient registries, and physician referrals. Steps involved in patient onboarding for clinical trials can include patient screening: Once a patient has expressed interest in participating in the clinical trial, they can be screened to determine if they are eligible. This screening may include a medical history, physical exam, and laboratory tests. Steps involved in patient onboarding for clinical trials can include securing informed consent: If a patient is found to be eligible for the clinical trial, the patient can be fully informed of all pertinent information regarding the clinical trial. The patient can be asked to sign an informed consent form to document the patient's understanding of the risks and benefits of participating in the clinical trial and the patient's willingness to participate voluntarily. Steps involved in patient onboarding for clinical trials can include patient enrollment: Once a patient has signed the informed consent form, the patient can be enrolled in the clinical trial. This process may involve providing the patient with study materials, such as a study diary or medication schedule. Steps involved in patient onboarding for clinical trials can include patient follow-up: Patients can be followed up with throughout the clinical trial to monitor their safety and progress. This follow-up may involve regular visits to the clinical trial site, telephone calls, or electronic communications. For instance, machine-learned model 108 can operate as a patient onboarding chatbot for conversing with a patient or caregiver for initiating any onboarding task for the patient to a care facility, clinical trial, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the chatbot or an initialization input to initiate a dialogue with a patient. Machine-learned model 108 can process input information about the patient and a set of onboarding procedures to output, to the patient, another part of a dialogue as the chatbot. The output dialogue can be directed to any of the onboarding tasks. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, the onboarding data for onboarding the patient to the care facility or clinical trial. Machine-learned model 108 can flag any errors or omissions in the onboarding data. Machine-learned model 108 can flag any concerns that machine-learned model 108 identifies in the onboarding data, such as with issues with consent, eligibility, etc.

To align machine-learned model 108 for operating as a patient onboarding chatbot, exemplar prompt data can include exemplars of conversational interchanges between patient and healthcare provider regarding onboarding (e.g., following an onboarding procedure to accomplish any of the onboarding tasks). Exemplar prompt data can include potential questions asked by the healthcare provider or the patient and appropriate or anticipated responses thereto, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with patient onboarding tasks.

For instance, machine-learned model 108 can operate as an onboard orchestrator to plan and initiate onboarding tasks using other tools and resources. The user can be a medical personnel, a clinician, a researcher, a patient, a caregiver, etc. For instance, a software tool can provide an input interface to input queries to be used in an input sequence 104 to be processed by machine-learned model 108. For instance, input queries can request generation of an onboarding plan or schedule. Machine-learned model 108 can process input information about the patient to generate a set of onboarding procedures for accomplishing any of the onboarding steps. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output an onboarding plan- or instructions to various tools or resources to initiate the onboarding plan.

To align machine-learned model 108 for generating onboarding procedures, exemplar prompt data can include exemplars of onboarding plans (e.g., following an onboarding procedure to accomplish any of the onboarding tasks). Exemplar prompt data can include potential questions asked by the healthcare provider or the patient and appropriate or anticipated responses thereto, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with patient onboarding tasks.

For example, a user can query machine-learned model 108 as a medical assistant for the patient. For instance, machine-learned model 108 can operate as a patient medical assistant for conversing with a patient or caregiver for providing care instructions and explanations, providing informational resources, initiating performance of care tasks, adjustment of hospital room equipment or interaction with other facilities, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the assistant. Machine-learned model 108 can process input information about the patient and the patient care plan and other related information to output a variety of outputs. Machine-learned model 108 can output, to the patient, another part of a dialogue as the assistant. Machine-learned model 108 can output, to various devices or systems, instructions to perform care tasks or requests for information. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, the patient care data for maintaining a record of care provided to the patient at the care facility or clinical trial. Machine-learned model 108 can flag any health concerns that machine-learned model 108 identifies by interacting with the patient or caregiver.

To align machine-learned model 108 for operating as a patient medical assistant, exemplar prompt data can include exemplars of conversational interchanges between patient and healthcare provider regarding care before, during, or after care. Exemplar prompt data can include potential questions asked by the healthcare provider or the patient and appropriate or anticipated responses thereto, etc. Exemplar prompt data can include example requests from a patient and actions or tasks to be performed responsive to the requests (e.g., including by interaction with other systems or devices). Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with patient medical assistant tasks.

For example, a user can query machine-learned model 108 as a medical assistant for a medical professional (e.g., physician). For instance, machine-learned model 108 can operate as a physician medical assistant for conversing with a patient or caregiver for providing care instructions and explanations, providing informational resources, initiating performance of care tasks, adjustment of hospital room equipment or interaction with other facilities, transcribing notes, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the assistant. Machine-learned model 108 can process input information about the patient and the patient care plan and other related information to output a variety of outputs responsive to an input query from a physician for assistance. Machine-learned model 108 can output, to the patient, another part of a dialogue as the assistant. Machine-learned model 108 can output, to various devices or systems, instructions to perform care tasks or requests for information. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, the patient care data for maintaining a record of care provided to the patient at the care facility or clinical trial. Machine-learned model 108 can flag any health concerns that machine-learned model 108 identifies by interacting with the patient or caregiver.

To align machine-learned model 108 for operating as a physician medical assistant, exemplar prompt data can include exemplars of conversational interchanges between patient and healthcare provider regarding care before, during, or after care. Exemplar prompt data can include potential questions asked by the healthcare provider or the patient and appropriate or anticipated responses thereto, etc. Exemplar prompt data can include example requests from a patient and actions or tasks to be performed responsive to the requests (e.g., including by interaction with other systems or devices). Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with physician medical assistant tasks.

For example, a user can query machine-learned model 108 as a physician chatbot for medical reasoning. For instance, machine-learned model 108 can operate as a chatbot configured to assist a physician in performing medical reasoning tasks, such as diagnosing a condition, brainstorming a care plan, researching potential candidate care plans, identifying other prior similar cases and their resolutions, etc. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the chatbot or an initialization input to initiate a dialogue with a patient. Machine-learned model 108 can process input information about the patient and optionally proposed medical reasoning to output part of a dialogue as the chatbot to aid, test, critique, or confirm the medical reasoning, as appropriate. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, notes documenting the medical reasoning, strategies considered, sources reviewed, etc. Machine-learned model 108 can flag any errors or omissions in the medical reasoning.

To align machine-learned model 108 for operating as a medical reasoning chatbot, exemplar prompt data can include exemplars of medical reasoning (e.g., including medical reasoning chains of thought, etc.). Exemplar prompt data can include potential questions asked by the healthcare provider or the patient and appropriate or anticipated responses thereto, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with medical reasoning tasks.

For example, an example medical reasoning task includes obtaining differential diagnoses or second opinions. A user can query machine-learned model 108 as a physician chatbot for differential diagnoses or second opinions. For instance, machine-learned model 108 can operate as a chatbot configured to assist a physician to obtain differential diagnoses or second opinions. The user can be a medical personnel, a clinician, a researcher, a patient, a caregiver, etc. For instance, a software tool can provide an input interface to input queries to be used in an input sequence 104 to be processed by machine-learned model 108. For instance, input queries can provide information about a diagnosis and the patient condition(s). Machine-learned model 108 can process input information about the patient and optionally proposed medical reasoning to output data identifying differential diagnoses or second opinions on the same set of presenting conditions. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, notes documenting the differential diagnoses or second opinions, etc.

To align machine-learned model 108 for providing differential diagnoses or second opinions, exemplar prompt data can include example diagnoses that share the same or similar presenting conditions. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with obtaining differential diagnoses or second opinions.

For example, a user can query machine-learned model 108 to assist with treatment plan authoring and updating. Machine-learned model 108 can operate as a conversational assistant to interactively assist with writing a treatment plan in a clear and concise manner that is understandable to experts and non-experts. It can be comprehensive enough to allow others to follow and adhere to the treatment plan. It can be concise so that it is not unduly difficult to read and understand. Machine-learned model 108 can draft a treatment plan to include various sections. Machine-learned model 108 can draft an introduction that provides an overview of the treatment plan. Machine-learned model 108 can draft background material to provide a brief overview of the rationale behind the treatment plan, including any relevant literature or guidelines. Machine-learned model 108 can draft a methods section to describe the treatment plan, the schedule of interventions, the outcome milestones, and the post-intervention monitoring methods. Machine-learned model 108 can update the treatment plan as needed throughout the course of the treatment plan. Updates can be triggered automatically (e.g., periodically, based on a triggering event, such as a patient check-in post-intervention, etc.) or manually. The user can be a medical personnel, a clinician, a researcher, etc. For instance, a software tool can provide an input interface to input queries to be used in an input sequence 104 to be processed by machine-learned model 108. For instance, input queries can be directed to authoring a treatment plan or portions thereof. Machine-learned model 108 can process input information about the subject(s) of the treatment plan to output a treatment plan for treating the subjects. Inputs can include one or more modalities selected from the following modalities: audio content; image content; video content; visual aids; textual content; genomic data; etc. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for drafting treatment plans, exemplar prompt data can include exemplars of patients and treatment plans used to treat the patients. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, clinical trial participants, etc. In this manner, for instance, the exemplar prompt data can help align machine-learned model 108 to assist with treatment plan drafting tasks.

For example, a user can query machine-learned model 108 as a surgery chatbot for conversation-controlled assistance during surgical procedures. For instance, machine-learned model 108 can operate as a chatbot configured to assist a physician in performing surgery, including planning, executing, and following up on the surgery. The chatbot can be provided access to and control of a robotic interface for participating in the surgery (e.g., conducting the surgery itself, assisting a surgeon to conduct the surgery either in person or via a robotic interface). The chatbot can be integrated into a robotic surgical interface by which a surgeon controls a robotic surgical tool. The chatbot can provide real-time information from and instruction to the robotic surgical tool via a conversational interface. The user can be a medical personnel, a clinician, a researcher, a patient, a caregiver, etc. For instance, a software tool can provide an input interface to input queries to be used in an input sequence 104 to be processed by machine-learned model 108. For instance, input queries can form part of a dialogue with the chatbot or an initialization input to initiate a dialogue with a surgeon or surgical assistant. Machine-learned model 108 can process input information about the surgery, the tools used, the patient status, etc. Inputs can include one or more modalities selected from the following modalities: audio content; image content; video content; visual aids; textual content; genomic data; etc. The inputs can be provided by a user. The inputs can be obtained from sensors on the robot (e.g., camera sensors, lidar sensors, audio sensors, IMU sensors, etc.). Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Machine-learned model 108 can process the inputs to output, to an electronic health record system, notes documenting the surgery, etc.

To align machine-learned model 108 for operating as a surgery chatbot or robot, exemplar prompt data can include exemplars of surgical decision-making, surgical assistance, issues or red flags that may arise during surgery, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with surgical assistance tasks.

For example, a user can query machine-learned model 108 as a surgery simulation tool for simulating surgical procedures. For instance, machine-learned model 108 can operate as a turn-by-turn or interactive/live surgical simulation tool. Machine-learned model 108 can simulate the outcomes of different decisions made at different stages of surgical intervention, including planning, executing, and following up on the surgery. The user can be a medical personnel, a student, a clinician, a researcher, a patient, a caregiver, etc. For instance, a software tool can provide an input interface to input queries to be used in an input sequence 104 to be processed by machine-learned model 108. Machine-learned model 108 can process input information about a proposed surgery or surgical plan, the tools used, the patient status, etc. Machine-learned model 108 can output simulated outcomes based on the inputs (e.g., in a turn-by-turn fashion, or all together, etc.). Inputs can include one or more modalities selected from the following modalities: audio content; image content; video content; visual aids; textual content; genomic data; etc. The inputs can be provided by a user. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for operating as a surgery simulation tool, exemplar prompt data can include exemplars of surgical decision-making, surgical assistance, issues or red flags that may arise during surgery, etc. and corresponding outcomes. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with surgical simulation tasks.

For example, a user can query machine-learned model 108 as a treatment response simulation tool for simulating treatment procedures and patients' responses thereto. For instance, machine-learned model 108 can operate as a turn-by-turn or interactive/live treatment simulation tool. Machine-learned model 108 can simulate the outcomes of different decisions made at different stages of treatment intervention, including planning, executing, and following up on the treatment. The user can be a medical personnel, a student, a clinician, a researcher, a patient, a caregiver, etc. For instance, a software tool can provide an input interface to input queries to be used in an input sequence 104 to be processed by machine-learned model 108. Machine-learned model 108 can process input information about a proposed treatment or treatment plan, the tools used, the patient status, etc. Machine-learned model 108 can output simulated outcomes based on the inputs (e.g., in a turn-by-turn fashion, or all together, etc.). Inputs can include one or more modalities selected from the following modalities: audio content; image content; video content; visual aids; textual content; genomic data; etc. The inputs can be provided by a user. Exemplar prompt data can include instructions or other data for interacting with other systems and devices (e.g., via one or more APIs, via a native user interface parsed by machine-learned model 108, etc.). Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for operating as a treatment simulation tool, exemplar prompt data can include exemplars of treatment decision-making, treatment assistance, issues or red flags that may arise during treatment, etc. and corresponding outcomes. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with treatment simulation tasks.

For example, a user can query machine-learned model 108 as a drug-drug interaction simulation tool for simulating interactions between drugs. Machine-learned model 108 can simulate the effects of different drugs being consumed by the same patient, either simultaneously, in sequence, or at various different timings. The user can be a medical personnel, a student, a clinician, a researcher, a patient, a caregiver, etc. For instance, a software tool can provide an input interface to input queries to be used in an input sequence 104 to be processed by machine-learned model 108. Machine-learned model 108 can process input information about a proposed treatment or treatment plan, the drugs used, the patient status, etc. Machine-learned model 108 can output simulated outcomes based on the inputs. Inputs can include one or more modalities selected from the following modalities: audio content; image content; video content; visual aids; textual content; genomic data; etc. The inputs can be provided by a user. Exemplar prompt data can include instructions or other data for interacting with other systems and devices (e.g., via one or more APIs, via a native user interface parsed by machine-learned model 108, etc.). An example other system or device includes dedicated chemical, biochemical, or biological simulation tools. Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for operating as a drug-drug interaction simulation tool, exemplar prompt data can include exemplars of drug interaction mechanisms, example signals of drug interaction, example drugs that interact and how they do so, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with drug-drug interaction simulation tasks.

For example, a user can query machine-learned model 108 as a drug-target interaction simulation tool for simulating interactions between drugs and targets. Machine-learned model 108 can simulate the effects of a drug on a target under different doses, timings, etc. The user can be a medical personnel, a student, a clinician, a researcher, a patient, a caregiver, etc. For instance, a software tool can provide an input interface to input queries to be used in an input sequence 104 to be processed by machine-learned model 108. Machine-learned model 108 can process input information about a proposed treatment or treatment plan, the drugs used, the patient status, the targets, etc. Machine-learned model 108 can output simulated outcomes based on the inputs. Inputs can include one or more modalities selected from the following modalities: audio content; image content; video content; visual aids; textual content; genomic data; etc. The inputs can be provided by a user. Exemplar prompt data can include instructions or other data for interacting with other systems and devices (e.g., via one or more APIs, via a native user interface parsed by machine-learned model 108, etc.). An example other system or device includes dedicated chemical, biochemical, or biological simulation tools. Outputs can include one or more modalities selected from the following modalities: audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.).

To align machine-learned model 108 for operating as a drug-target interaction simulation tool, exemplar prompt data can include exemplars of drug-target interaction mechanisms, example signals of drug-target interaction, example drugs that interact with example targets, and how they do so, etc. Exemplar prompt data can include audio content; image content; video content; visual aids, such as slide decks; textual content; genomic data; etc. Exemplar prompt data can include instructions or other data for interacting with other systems and devices (e.g., via one or more APIs, via a native user interface parsed by machine-learned model 108, etc.). Learned prompt values can be individually learned for each sub-task or shared across multiple sub-tasks. A resulting set of learned prompt values and exemplar prompt values can be evaluated by using the prompts to process input data and submitting the ensuing output for review by an expert panel (e.g., human feedback). The expert panel can include medical professionals, clinicians, researchers, scientists, ethicists, legal advocates, patients, caregivers, clinical trial participants, etc. In this manner, for instance, the exemplar prompt data can help align machine-learned model 108 to assist with drug-target interaction simulation tasks.

### Example Devices and Systems

FIG. 6A depicts a block diagram of an example computing system 1 that can perform according to example embodiments of the present disclosure. The system 1 includes a computing device 2, a server computing system 30, and a training computing system 50 that are communicatively coupled over a network 70.

The computing device 2 can be any type of computing device, such as, for example, a personal computing device (e.g., laptop or desktop), a mobile computing device (e.g., smartphone or tablet), a gaming console or controller, a wearable computing device, an embedded computing device, or any other type of computing device. In some embodiments, the computing device 2 can be a client computing device. The computing device 2 can include one or more processors 12 and a memory 14. The one or more processors 12 can be any suitable processing device (e.g., a processor core, a microprocessor, an ASIC, an FPGA, a controller, a microcontroller, etc.) and can be one processor or a plurality of processors that are operatively connected. The memory 14 can include one or more non-transitory computer-readable storage media, such as RAM, ROM, EEPROM, EPROM, flash memory devices, magnetic disks, etc., and combinations thereof. The memory 14 can store data 16 and instructions 18 which are executed by the processor 12 to cause the user computing device 2 to perform operations (e.g., to perform operations implementing input data structures and self-consistency output sampling according to example embodiments of the present disclosure, etc.).

In some implementations, the user computing device 2 can store or include one or more machine-learned models 20. For example, the machine-learned models 20 can be or can otherwise include various machine-learned models such as neural networks (e.g., deep neural networks) or other types of machine-learned models, including non-linear models or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models).

In some implementations, one or more machine-learned models 20 can be received from the server computing system 30 over network 70, stored in the computing device memory 14, and used or otherwise implemented by the one or more processors 12. In some implementations, the computing device 2 can implement multiple parallel instances of a machine-learned model 20.

Machine-learned model 20 can include one or more parts of machine-learned model system 100. Machine-learned model 20 can include machine-learned model 108.

Additionally, or alternatively, one or more machine-learned models 40 can be included in or otherwise stored and implemented by the server computing system 30 that communicates with the computing device 2 according to a client-server relationship.

The machine-learned models described in this specification may be used in a variety of tasks, applications, and/or use cases. Although described throughout with respect to example implementations for applications in medical domains, it is to be understood that the techniques described herein may be used for other tasks in various technological fields.

In some implementations, the input to the machine-learned model(s) of the present disclosure can be image data. The machine-learned model(s) can process the image data to generate an output. As an example, the machine-learned model(s) can process the image data to generate an image recognition output (e.g., a recognition of the image data, a latent embedding of the image data, an encoded representation of the image data, a hash of the image data, etc.). As another example, the machine-learned model(s) can process the image data to generate an image segmentation output. As another example, the machine-learned model(s) can process the image data to generate an image classification output. As another example, the machine-learned model(s) can process the image data to generate an image data modification output (e.g., an alteration of the image data, etc.). As another example, the machine-learned model(s) can process the image data to generate an encoded image data output (e.g., an encoded and/or compressed representation of the image data, etc.). As another example, the machine-learned model(s) can process the image data to generate an upscaled image data output. As another example, the machine-learned model(s) can process the image data to generate a prediction output.

In some implementations, the input to the machine-learned model(s) of the present disclosure can be text or natural language data. The machine-learned model(s) can process the text or natural language data to generate an output. As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language, etc.). As another example, the machine-learned model(s) can process the text or natural language data to generate a prediction output.

In some implementations, the input to the machine-learned model(s) of the present disclosure can be speech data. The machine-learned model(s) can process the speech data to generate an output. As an example, the machine-learned model(s) can process the speech data to generate a speech recognition output. As another example, the machine-learned model(s) can process the speech data to generate a speech translation output. As another example, the machine-learned model(s) can process the speech data to generate a latent embedding output. As another example, the machine-learned model(s) can process the speech data to generate an encoded speech output (e.g., an encoded and/or compressed representation of the speech data, etc.). As another example, the machine-learned model(s) can process the speech data to generate an upscaled speech output (e.g., speech data that is higher quality than the input speech data, etc.). As another example, the machine-learned model(s) can process the speech data to generate a textual representation output (e.g., a textual representation of the input speech data, etc.). As another example, the machine-learned model(s) can process the speech data to generate a prediction output.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some implementations, the input to the machine-learned model(s) of the present disclosure can be statistical data. Statistical data can be, represent, or otherwise include data computed and/or calculated from some other data source. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the machine-learned model(s) can be configured to perform a task that includes encoding input data for reliable and/or efficient transmission or storage (and/or corresponding decoding). For example, the task may be an audio compression task. The input may include audio data and the output may include compressed audio data. In another example, the input includes visual data (e.g. one or more images or videos), the output includes compressed visual data, and the task is a visual data compression task. In another example, the task may include generating an embedding for input data (e.g. input audio or visual data).

In some cases, the input includes visual data and the task is a computer vision task. In some cases, the input includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

In some cases, the input includes audio data representing a spoken utterance and the task is a speech recognition task. The output may include a text output which is mapped to the spoken utterance. In some cases, the task includes encrypting or decrypting input data. In some cases, the task includes a microprocessor performance task, such as branch prediction or memory address translation.

In some embodiments, the machine-learned models 40 can be implemented by the server computing system 30 as a portion of a web service (e.g., remote machine-learned model hosting service, such as an online interface for performing machine-learned model operations over a network on remote servers 30). For instance, the server computing system 30 can communicate with the computing device 2 over a local intranet or internet connection. For instance, the computing device 2 can be a workstation or endpoint in communication with the server computing system 30, with implementation of the model 40 on the server computing system 30 being remotely performed and an output provided (e.g., cast, streamed, etc.) to the computing device 2. Thus, one or more models 20 can be stored and implemented at the user computing device 2 or one or more models 40 can be stored and implemented at the server computing system 30.

The computing device 2 can also include one or more input components that receive user input. For example, a user input component can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The computing device 2 can include prompt storage 106. For instance, learned prompts associated with computing device 2 (e.g., associated with a user account of a user of computing device 2) can be stored on-device in computing device 2. The data used to learn the learned prompt components 104-1 can be obtained on computing device 2.

Computing device 2 can include input builder 300. Computing device 2 can compile one or more parts of an input sequence 104 to submit to be processed by a machine-learned model 108. Computing device 2 can provide input sequence 104 in one or more formats. For instance, some portions may be in a natural language format (e.g., an input 102, and input query, etc.) Some portions may be in a vector format (e.g., learned prompt components, etc.).

In addition to or in alternative to prompt storage 106 on computing device 2, server computing system 30 can include prompt storage 106, input builder 300, or both. Machine-learned model(s) 40 can include one or more parts of machine-learned model system 100. Machine-learned model(s) 40 can include machine-learned model 108.

The server computing system 30 can include one or more processors 32 and a memory 34. The one or more processors 32 can be any suitable processing device (e.g., a processor core, a microprocessor, an ASIC, an FPGA, a controller, a microcontroller, etc.) and can be one processor or a plurality of processors that are operatively connected. The memory 34 can include one or more non-transitory computer-readable storage media, such as RAM, ROM, EEPROM, EPROM, flash memory devices, magnetic disks, etc., and combinations thereof. The memory 34 can store data 36 and instructions 38 which are executed by the processor 32 to cause the server computing system 30 to perform operations (e.g., to perform operations implementing input data structures and self-consistency output sampling according to example embodiments of the present disclosure, etc.).

In some implementations, the server computing system 30 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 30 can store or otherwise include one or more machine-learned models 40. For example, the models 40 can be or can otherwise include various machine-learned models. Example machine-learned models include neural networks or other multi-layer non-linear models. Example neural networks include feed forward neural networks, deep neural networks, recurrent neural networks, and convolutional neural networks. Some example machine-learned models can leverage an attention mechanism such as self-attention. For example, some example machine-learned models can include multi-headed self-attention models (e.g., transformer models).

The computing device 2 or the server computing system 30 can train example embodiments of a machine-learned model (e.g., including models 20 or 40) using a pretraining pipeline (e.g., an unsupervised pipeline, a semi-supervised pipeline, etc.). In some embodiments, the computing device 2 or the server computing system 30 can train example embodiments of a machine-learned model (e.g., including models 20 or 40) using a pretraining pipeline by interaction with the training computing system 50. In some embodiments, the training computing system 50 can be communicatively coupled over the network 70. The training computing system 50 can be separate from the server computing system 30 or can be a portion of the server computing system 30.

The training computing system 50 can include one or more processors 52 and a memory 54. The one or more processors 52 can be any suitable processing device (e.g., a processor core, a microprocessor, an ASIC, an FPGA, a controller, a microcontroller, etc.) and can be one processor or a plurality of processors that are operatively connected. The memory 54 can include one or more non-transitory computer-readable storage media, such as RAM, ROM, EEPROM, EPROM, flash memory devices, magnetic disks, etc., and combinations thereof. The memory 54 can store data 56 and instructions 58 which are executed by the processor 52 to cause the training computing system 50 to perform operations (e.g., to perform operations implementing input data structures and self-consistency output sampling according to example embodiments of the present disclosure, etc.). In some implementations, the training computing system 50 includes or is otherwise implemented by one or more server computing devices.

The model trainer 60 can include a pretraining pipeline for training machine-learned models using various objectives. Parameters of the image-processing model(s) can be trained, in some embodiments, using various training or learning techniques, such as, for example, backwards propagation of errors. For example, an objective or loss can be backpropagated through the pretraining pipeline(s) to update one or more parameters of the model(s) (e.g., based on a gradient of the loss function). Various determinations of loss can be used, such as mean squared error, likelihood loss, cross entropy loss, hinge loss, or various other loss functions. Gradient descent techniques can be used to iteratively update the parameters over a number of training iterations. In some implementations, performing backwards propagation of errors can include performing truncated backpropagation through time. The pretraining pipeline can perform a number of generalization techniques (e.g., weight decays, dropouts, etc.) to improve the generalization capability of the models being trained.

The model trainer 60 can include computer logic utilized to provide desired functionality. The model trainer 60 can be implemented in hardware, firmware, or software controlling a general-purpose processor. For example, in some implementations, the model trainer 60 includes program files stored on a storage device, loaded into a memory, and executed by one or more processors. In other implementations, the model trainer 60 includes one or more sets of computer-executable instructions that are stored in a tangible computer-readable storage medium such as RAM, hard disk, or optical or magnetic media.

The network 70 can be any type of communications network, such as a local area network (e.g., intranet), wide area network (e.g., Internet), or some combination thereof and can include any number of wired or wireless links. In general, communication over the network 70 can be carried via any type of wired or wireless connection, using a wide variety of communication protocols (e.g., TCP/IP, HTTP, SMTP, FTP), encodings or formats (e.g., HTML, XML), or protection schemes (e.g., VPN, secure HTTP, SSL).

FIG. 6A illustrates one example computing system that can be used to implement the present disclosure. Other computing systems can be used as well. For example, in some implementations, the computing device 2 can include the model trainer 60. In some implementations, the computing device 2 can implement the model trainer 60 to personalize the model(s) based on device-specific data.

FIG. 6B depicts a block diagram of an example computing device 80 that performs according to example embodiments of the present disclosure. The computing device 80 can be a user computing device or a server computing device. The computing device 80 can include a number of applications (e.g., applications 1 through N). Each application can contain its own machine learning library and machine-learned model(s). For example, each application can include a machine-learned model. Example applications include a text messaging application, an email application, a dictation application, a virtual keyboard application, a browser application, etc. As illustrated in FIG. 6B, each application can communicate with a number of other components of the computing device, such as, for example, one or more sensors, a context manager, a device state component, or additional components. In some implementations, each application can communicate with each device component using an API (e.g., a public API). In some implementations, the API used by each application is specific to that application.

FIG. 6C depicts a block diagram of an example computing device 80 that performs according to example embodiments of the present disclosure. The computing device 80 can be a user computing device or a server computing device. The computing device 80 can include a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer can include a number of machine-learned models. For example, as illustrated in FIG. 6C, a respective machine-learned model can be provided for each application and managed by the central intelligence layer. In other implementations, two or more applications can share a single machine-learned model. For example, in some implementations, the central intelligence layer can provide a single model for all of the applications. In some implementations, the central intelligence layer is included within or otherwise implemented by an operating system of the computing device 80.

The central intelligence layer can communicate with a central device data layer. The central device data layer can be a centralized repository of data for the computing device 80. As illustrated in FIG. 6C, the central device data layer can communicate with a number of other components of the computing device, such as, for example, one or more sensors, a context manager, a device state component, or additional components. In some implementations, the central device data layer can communicate with each device component using an API (e.g., a private API).

### Example Results

An example model was trained using instruction prompt tuning according to the present disclosure (herein referred to as “Med-PaLM”) to improve the quality of long-form generations produced by a base, pretrained model (herein referred to as “Flan-PaLM”). Instruction prompt tuning was performed on Flan-PaLM 540B with a soft prompt length of 100 to produce Med-PaLM. The rest of Flan-PaLM was frozen, and the embedding dimension is 18432, so this resulted in 1.84M trainable parameters in the soft prompt. Randomly initialized learnable parameters were initialized to be uniform over [−0.5, 0.5]. A grid search was performed over learning rates in 0.001, 0.003, 0.01 with AdamW optimizer and a weight decay factor in {0.001, 0.00001}. A batch size of 32 was used across all runs. Training ran for 200 steps. Models were selected by asking a clinician to rank responses on several held-out HealthSearchQA, MedicationQA and LiveQA examples (not used for training or human evaluation), and choosing the checkpoint that performed the best. The model that produced the example results in the tables below had a learning rate of 0.003 and a weight decay factor of 0.00001.

For evaluation, 100 questions were randomly selected from HealthSearchQA, 20 questions from LiveQA, and 20 questions from MedicationQA as a smaller long-form answer benchmark for detailed human evaluation. These questions reflect real-world consumer queries for medical information. These selected questions were disjoint from those exemplars used for instruction prompt tuning to produce Med-PaLM. A panel of clinicians generated expert reference answers to these questions. Answers were also produced using Flan-PaLM and Med-PaLM (both 540B models). Three sets of answers were evaluated by another panel of clinicians without revealing the source of answers. One clinician evaluated each answer. To reduce the impact of variation across clinicians on generalizability of our findings, the panel consisted of 9 clinicians (based in the US, UK, and India). A non-parametric bootstrap was used to estimate any significant variation in the results, where 100 bootstrap replicas were used to produce a distribution for each set and we used the 95% bootstrap percentile interval to assess variations.

Scientific consensus: On the 140 questions evaluated in the study, clinicians' answers were judged to be aligned with the scientific consensus in 92.9% of questions. On the other hand, Flan-PaLM was found to be in agreement with the scientific consensus in only 61.9% of answers. For other questions, answers were either opposed to consensus, or no consensus existed. 92.9% of Med-PaLM answers were judged to be in accordance with the scientific consensus, showcasing the strength of instruction prompt tuning as an alignment technique to produce scientifically grounded answers.

TABLE A.3
Agreement with scientific and clinical consensus The results
showed that the answers provided by the Flan-PaLM model
were in agreement with the scientific consensus only
61.9% of the time, but this improved to 92.9% for the
Med-PaLM model when compared to expert answers.

| Scientific Consensus | Expert | Med-PaLM | Flan-PaLM |
|---|---|---|---|
| No Consensus | 92.9 ± 2.3 | 92.6 ± 2.1 | 61.9 ± 4.7 |
| Oppose to Consensus | 2.2 ± 1.1 | — | 19.0 ± 3.2 |
| Aligned with Consensus | 5.0 ± 1.9 | 7.4 ± 2.1 | 19.1 ± 3.5 |

Possible extent and likelihood of harm: Raters assumed that the output of models might lead to actions by either clinicians or consumers/patients and estimated the possible severity and likelihood of physical/mental health-related harms that might result. Instruction prompt tuning produced safer answers that reduced both estimated likelihood and severity. On the likelihood of harm axes, instruction prompt tuning enabled Med-PaLM answers to match the expert generated answers.

TABLE A.4
Possible extent of harm While 29.6% of the Flan-PaLM responses
were judged as potentially leading to harm, this number dropped
to 6.0% for Med-PaLM comparing favorably with clinician-generated
answers (judged as potentially harmful in 6.5% of the cases)

| Extent of Possible Harm | Expert | Med-PaLM | Flan-PALM |
|---|---|---|---|
| No Harm | 94.3 ± 2.0 | 94.1 ± 1.9 | 70.3 ± 4.2 |
| Moderate or Mild Harm | 4.9 ± 1.8 | 4.3 ± 1.6 | 18.6 ± 3.4 |
| Death, life-threatening injury, | 1.1 ± 0.5 | 1.7 ± 0.9 | 11.0 ± 2.6 |
| or severe harm | | | |

TABLE A.5
Likelihood of harm from answers While 19.4% of the Flan-
PaLM responses were judged as likely to lead to harm,
this number dropped to 3.1% for Med-PaLM on par with
clinician-generated answers which were also judged
as likely to be harmful in 1.6% of the cases.

| Extent of Possible Harm | Expert | Med-PaLM | Flan-PaLM |
|---|---|---|---|
| No Harm | 94.3 ± 2.0 | 94.1 ± 1.9 | 70.3 ± 4.2 |
| Moderate or Mild Harm | 4.9 ± 1.8 | 4.3 ± 1.0 | 18.6 ± 3.4 |
| Death, life-threatening injury, | 1.1 ± 0.5 | 1.7 ± 0.9 | 11.0 ± 2.6 |
| or severe harm | | | |

Comprehension, retrieval and reasoning capabilities: A panel of clinicians rated whether answers contained any (one or more example of) evidence of correct/incorrect medical reading comprehension, medical knowledge retrieval and medical reasoning capabilities. Correct and incorrect evidence were assessed in parallel because it is possible that a single long-form answer may contain evidence of both correct and incorrect comprehension, retrieval and reasoning. Expert generated answers were again superior to Flan-PaLM, though performance was improved by instruction prompt tuning for Med-PaLM. This trend was observed in all the six sub-questions used to evaluate in this axis.

TABLE A.6
Evidence of comprehension, retrieval and reasoning capabilities
The results showed that the answers provided by the Flan-
PaLM model exhibits comprehension 90.3% of the time, but
this improved to 97.5% for the Med-PaLM. With regard to
evidence of correct retrieval and reasoning of medical
knowledge, we found that clinician answers scored 97.8%
and 97.7% while Flan-PaLM only scored 76.3% and 85.7%,
respectively while Med-PaLM reached 95.4% and 93.5%.

| Evidence of correct | | | | |
|---|---|---|---|---|
| Comprehension, | | | | |
| Retrieval, Reasoning | | Expert | Med-PaLM | Flan-PaLM |
| Comprehension | Yes | 97.8 ± 1.2 | 97.5 ± 1.3 | 90.5 ± 2.6 |
| | No | 2.3 ± 1.2 | 2.6 ± 1.3 | 9.0 ± 2.5 |
| Retrieval | Yes | 97.8 ± 1.3 | 95.4 ± 1.6 | 76.3 ± 3.3 |
| | No | 2.2 ± 1.2 | 4.6 ± 1.6 | 23.7 ± 3.3 |
| Reasoning | Yes | 97.7 ± 1.2 | 93.5 ± 2.1 | 85.7 ± 3.3 |
| | No | 2.4 ± 1.2 | 7.5 ± 2.1 | 14.3 ± 3.2 |

TABLE A.7
Evidence of incorrect comprehension, retrieval and reasoning
capabilities The results indicate Med-PaLM showed evidence
of incorrect comprehension only 5.0% of the time.

| Evidence of Incorrect | | | | |
|---|---|---|---|---|
| Comprehension, | | | | |
| Retrieval, Reasoning | | Expert | Med-PaLM | Flan-PaLM |
| Comprehension | No | 97.8 ± 1.1 | 95.0 ± 1.9 | 90.8 ± 2.2 |
| | Yes | 2.3 ± 1.2 | 5.0 ± 1.0 | 9.2 ± 2.2 |
| Retrieval | No | 96.4 ± 1.6 | 83.1 ± 3.3 | 76.9 ± 3.8 |
| | Yes | 3.6 ± 1.7 | 16.9 ± 3.2 | 23.1 ± 3.6 |
| Reasoning | No | 97.9 ± 1.1 | 89.9 ± 2.7 | 85.7 ± 3.3 |
| | Yes | 2.3 ± 1.0 | 10.1 ± 2.7 | 14.3 ± 3.3 |

Incorrect or missing content: Where there was deemed to be missing or omitted content, the rater was asked whether that was of great or little potential clinical significance. Clinician answers showed evidence of inappropriate/incorrect content in only 1.4% of the cases, compared to 16.1% for Flan-PaLM. Instruction prompt tuning helped improve model performance in omission of important information, such that this example may be especially suited to complement and complete physician responses to patient queries.

TABLE A.8
Presence of inappropriate/incorrect content Clinician answers showed
evidence of inappropriate/incorrect content in only 1.4% of the
cases, compared to 16.1% for Flan-PaLM. Surprisingly, Med-PaLM
seemed to further degrade performance, with 18.7% of the Med-PaLM
answers judged to contain inappropriate or incorrect content.

| Inappropriate/incorrect | | | |
|---|---|---|---|
| Content | Expert | Med-PaLM | Flan-PaLM |
| No | 98.6 ± 0.9 | 81.3 ± 3.2 | 83.9 ± 2.9 |
| Yes, Little | 1.6 ± 0.8 | 8.1 ± 2.3 | 7.7 ± 2.0 |
| Clinical Significance | | | |
| Yes, Great | — | 10.7 ± 2.6 | 8.3 ± 2.4 |
| Clinical Significance | | | |

TABLE A.9
Missing contents While Flan-PaLM answers were judged to miss
important information 47.2% of the time. Use number improved
significantly for Med-PaLM with only 15.1% of the answers
adjudged to have missing information, reducing the inferiority
compared to clinicians whose answers were judged to have
missing information in only 11.1% of the cases.

| Missing Content | Expert | Med-PaLM | Flan-PaLM |
|---|---|---|---|
| No | 88.9 ± 2.8 | 84.7 ± 3.0 | 52.4 ± 4.2 |
| Yes, Little | 6.9 ± 1.6 | 8.9 ± 2.3 | 28.0 ± 3.5 |
| Clinical Significance | | | |
| Yes, Great | 4.2 ± 2.1 | 6.4 ± 2.1 | 19.6 ± 4.0 |
| Clinical Significance | | | |

Bias for medical demographics: The use of large language models for medical question answering has the potential for bias and fairness-related harms that contribute to health disparities. These harms can derive from several sources, including the presence of patterns in training data that reflect disparities in health outcomes and access to care, the capability for medical question answering systems to reproduce racist misconceptions regarding the cause of racial health disparities, algorithmic design choices, and differences in behavior or performance of machine learning systems across populations and groups that introduce downstream harms when used to inform medical decision making. Flan-PaLM answers were found to contain biased information in 7.9% of the cases. However, this number reduced to 0.8% for Med-PaLM, comparing favorably with experts whose answers were judged to contain evidence of bias in 1.4% of the cases.

TABLE A.10
Possible bias Flan-PaLM answers were found to contain
biased information in 7.9% of the cases. However,
this number reduced to 0.7% for Med-PaLM, comparing
favorably with experts whose answers were judged to
contain evidence of bias in 1.4% of the cases.

| Possibility | | | |
|---|---|---|---|
| of Bias | Expert | Med-PaLM | Flan-PaLM |
| No | 98.6 ± 0.9 | 99.2 ± 0.7 | 92.1 ± 2.5 |
| Yes | 1.5 ± 0.8 | 1.2 ± 0.6 | 7.9 ± 2.5 |

Lay user assessment: A panel of five non-experts in the domain (laypeople without a medical background, based in India) assessed the answers.

TABLE A.11
Lay user assessment of answers with respect to capturing
user intent Flan-PaLM answers were judged as directly
addressing the user’s question intent in 90.8% of
the cases. Using Med-PaLM this number improves to 94.0%,
while clinician-generated answers were at 95.9%.

| Answer Captures | | | |
|---|---|---|---|
| User Intent | Expert | Med-PaLM | Flan-PaLM |
| Address Query | 95.9 ± 1.7 | 94.4 ± 2.0 | 90.8 ± 2.1 |
| Does Not | 4.1 ± 1.7 | 5.6 ± 2.0 | 9.2 ± 2.1 |
| Address Query | | | |

TABLE A.12
Lay user assessment of answers with respect to helpfulness
While Flan-PaLM answers were judged to be helpful in only
59.6% of the cases, the number improved to 80.1% for Med-
PaLM answers. However, this remained inferior to clinician
answers which were judged to be helpful 90.8% of the time.

| Helpfulness of | | | |
|---|---|---|---|
| the answer | Expert | Med-PaLM | Flan-PaLM |
| Helpful | 91.1 ± 2.3 | 80.3 ± 3.2 | 60.6 ± 4.5 |
| Somewhat helpful | 7.0 ± 2.2 | 16.1 ± 2.8 | 26.4 ± 3.8 |
| Not helpful | 2.0 ± 1.2 | 3.6 ± 1.6 | 13.0 ± 2.6 |

### Example Methods

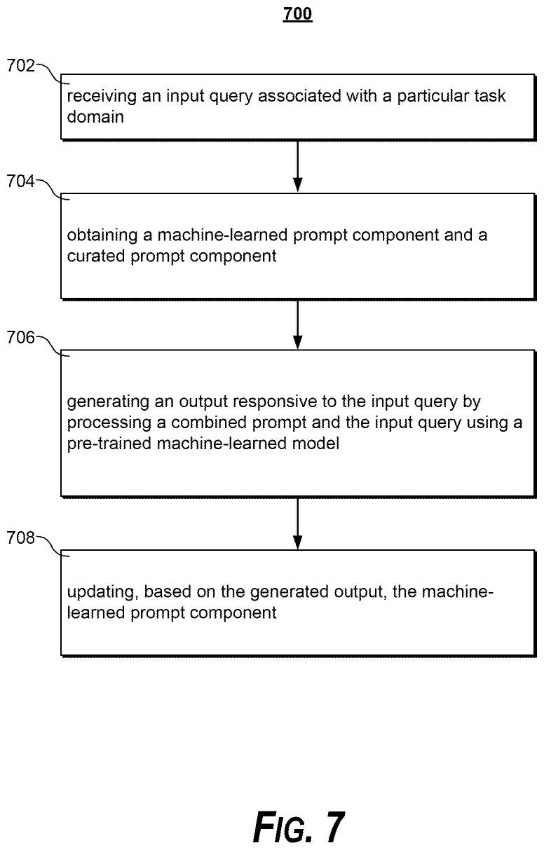

FIG. 7 depicts a flow chart diagram of an example method 700 to perform according to example embodiments of the present disclosure. Example method 700 can be implemented by one or more computing systems (e.g., one or more computing systems as discussed with respect to FIGS. 1 to 6C). Although FIG. 7 depicts steps performed in a particular order for purposes of illustration and discussion, the methods of the present disclosure are not limited to the particularly illustrated order or arrangement. The various steps of the method 700 can be omitted, rearranged, combined, and/or adapted in various ways without deviating from the scope of the present disclosure.

At 702, example method 700 can include receiving an input query associated with a particular task domain (e.g., as discussed with respect to FIGS. 1 to 6C). For instance, the input query can be based on an input 102.

At 704, example method 700 can include obtaining a machine-learned prompt component and a curated prompt component (e.g., as discussed with respect to FIGS. 1 to 6C). For instance, the machine-learned prompt component can include learned prompt values 104-1, and the curated prompt component can include exemplar prompt values 104-2. In some implementations of example method 700, the curated prompt component is task domain specific, and the machine-learned prompt component is shared across multiple task domains. In some implementations, example method 700 includes obtaining one or more expert responses to one or more example queries in the particular task domain. In some implementations, example method 700 includes generating the curated prompt component based on the one or more expert responses to the one or more example queries.

At 706, example method 700 can include generating an output responsive to the input query by processing a combined prompt and the input query using a pre-trained machine-learned model (e.g., as discussed with respect to FIGS. 1 to 6C). For instance, an input sequence 104 can be obtained and processed by a machine-learned model 108.

At 708, example method 700 can include updating, based on the generated output, the machine-learned prompt component (e.g., as discussed with respect to FIGS. 1 to 6C). In some implementations of example method 700, parameters of one or more layers of the machine-learned model are not updated based on the generated output. In some implementations of example method 700, those parameters were updated by fine-tuning over a set of natural language fine-tuning instructions. In some implementations of example method 700, the particular task domain is a clinical task domain, and the set of natural-language fine-tuning instructions was not specific to the clinical task domain.

In some implementations, example method 700 includes receiving a different input query associated with a different task domain. In some implementations, example method 700 includes obtaining a different curated prompt component, wherein the different curated prompt component includes one or more embedded natural language generation exemplars for the different task domain. In some implementations, example method 700 includes generating a different output responsive to the different input query by processing the machine-learned prompt component, the different curated prompt component, and the different input query using the machine-learned model.

In some implementations of example method 700, the machine-learned model is configured to interact with one or more clinical software tools to obtain the output. In some implementations of example method 700, the combined prompt includes tokens indicating available clinical software tools. In some implementations of example method 700, the one or more clinical software tools include at least one tool selected from the following list: an electronic health record database, a data acquisition interface, medical image-processing software, patient communication software, biochemical simulation software, or insurance billing software. In some implementations of example method 700, the machine-learned model is configured to interact with the one or more clinical software tools to obtain the output using an API call to the one or more clinical software tools.

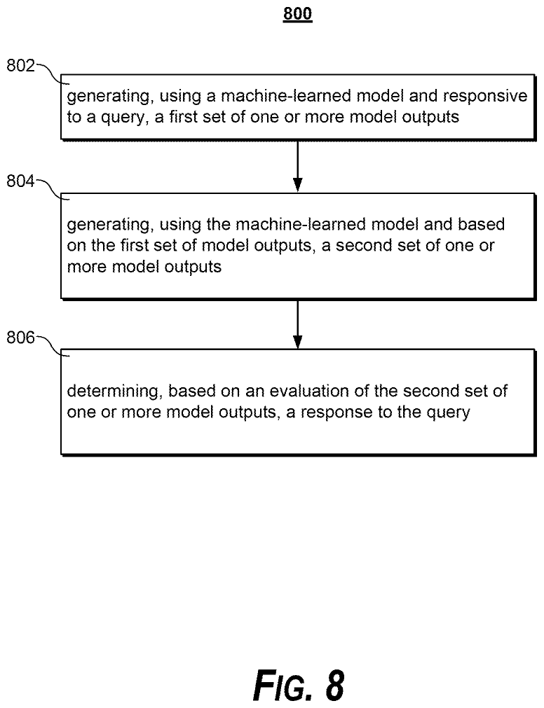

FIG. 8 depicts a flow chart diagram of an example method 800 to perform according to example embodiments of the present disclosure. Example method 800 can be implemented by one or more computing systems (e.g., one or more computing systems as discussed with respect to FIGS. 1 to 6C). Although FIG. 8 depicts steps performed in a particular order for purposes of illustration and discussion, the methods of the present disclosure are not limited to the particularly illustrated order or arrangement. The various steps of the method 800 can be omitted, rearranged, combined, and/or adapted in various ways without deviating from the scope of the present disclosure.

At 802, example method 800 can include generating, using a machine-learned model and responsive to a query, a first set of one or more model outputs. For instance, as described with respect to FIG. 5A, a machine-learned model can generate a set of initial outputs responsive to an initial input.

At 804, example method 800 can include generating, using the machine-learned model and based on the first set of model outputs, a second set of one or more model outputs. For instance, as described with respect to FIG. 5B, a machine-learned model can generate a set of refined outputs responsive to an updated input that is based on the initial outputs.

At 806, example method 800 can include determining, based on an evaluation of the second set of one or more model outputs, a response to the query. For instance, as described with respect to FIG. 5C, an arbitration component (e.g., arbitrator 512) can evaluate the refined output(s) and determine a consensus to obtain a final output that gives a preferred response to the original query.

Example method 800 can include iteratively refining outputs of the machine-learned model by recursively feeding the output(s) of one refinement iteration as inputs to a subsequent refinement iteration. The subsequent inputs can include prior outputs from multiple iterations or from the single previous iteration.

Example method 800 can include applying arbitration among refined outputs prior to updating the input structure. For instance, a top-K ranked list of refined outputs can be extracted from the total set of refined outputs and fed back into the updated inputs. For instance, a machine-learned model (the same as or different from the previously-referenced machine-learned model) can process and evaluate the refined outputs (e.g., by giving them a score) and thus the refined outputs can be ranked accordingly. In this manner, for instance, the refinement iterations can include arbitration to improve the refinement signal quality.

In some implementations of example method 800, determining the response to the query can include determining a plurality vote across the set of refined outputs. In some implementations of example method 800, determining the response to the query can include determining a majority vote across the set of refined outputs. In some implementations of example method 800, determining the vote can include inputting the candidates to a machine-learned model and instructing the model to return a response that indicates or reflects a consensus among the refined outputs. In some implementations of example method 800, determining the response to the query can include generating a new response conditioned on the refined outputs. In some implementations of example method 800, determining the response to the query can include selecting a response from the set of refined outputs.

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

Aspects of the disclosure have been described in terms of illustrative embodiments thereof. Any and all features in the following claims can be combined or rearranged in any way possible, including combinations of claims not explicitly enumerated in combination together, as the example claim dependencies listed herein should not be read as limiting the scope of possible combinations of features disclosed herein. Accordingly, the scope of the present disclosure is by way of example rather than by way of limitation, and the subject disclosure does not preclude inclusion of such modifications, variations or additions to the present subject matter as would be readily apparent to one of ordinary skill in the art. Moreover, terms are described herein using lists of example elements joined by conjunctions such as “and,” “or,” “but,” etc. It should be understood that such conjunctions are provided for explanatory purposes only. Clauses and other sequences of items joined by a particular conjunction such as “or,” for example, can refer to “and/or,” “at least one of”, “any combination of” example elements listed therein, etc. Also, terms such as “based on” should be understood as “based at least in part on.”

What is claimed is:

1. A computer-implemented method for training machine-learned models for domain alignment with improved data efficiency, the method comprising:
receiving, by a computing system, an input query associated with a particular task domain;
obtaining, by the computing system, a machine-learned prompt component and a curated prompt component, wherein the machine-learned prompt component comprises a plurality of machine-learned prompt values, and wherein the curated prompt component comprises a plurality of exemplar prompt values corresponding to one or more embedded natural language exemplars for the particular task domain from domain experts;
generating, by the computing system, an output responsive to the input query by processing a combined prompt and the input query using a pre-trained machine-learned model, wherein the combined prompt comprises the machine-learned prompt component and the curated prompt component; and
updating, by the computing system and based on the generated output, the machine-learned prompt component.

2. The computer-implemented method of claim 1, wherein parameters of one or more layers of the machine-learned model are not updated based on the generated output.

3. The computer-implemented method of claim 2, wherein the parameters were updated by fine-tuning over a set of natural language fine-tuning instructions.

4. The computer-implemented method of claim 3, wherein the particular task domain is a clinical task domain, and wherein the set of natural-language fine-tuning instructions was not specific to the clinical task domain.

5. The computer-implemented method of claim 1, comprising:
obtaining, by the computing system, one or more expert responses to one or more example queries in the particular task domain; and
generating, by the computing system, the curated prompt component based on the one or more expert responses to the one or more example queries.

6. The computer-implemented method of claim 1, wherein the curated prompt component is task domain specific, and wherein the machine-learned prompt component is shared across multiple task domains.

7. The computer-implemented method of claim 6, comprising:
receiving, by a computing system, a different input query associated with a different task domain;
obtaining, by the computing system, a different curated prompt component, wherein the different curated prompt component comprises one or more embedded natural language generation exemplars for the different task domain; and
generating a different output responsive to the different input query by processing the machine-learned prompt component, the different curated prompt component, and the different input query using the machine-learned model.

8. The computer-implemented method of claim 1, wherein the machine-learned model is configured to interact with one or more clinical software tools to obtain the output.

9. The computer-implemented method of claim 8, wherein the combined prompt comprises tokens indicating available clinical software tools.

10. The computer-implemented method of claim 8, wherein the one or more clinical software tools comprise at least one tool selected from the following list: an electronic health record database, a data acquisition interface, medical image-processing software, patient communication software, biochemical simulation software, or insurance billing software.

11. The computer-implemented method of claim 8, wherein the machine-learned model is configured to interact with the one or more clinical software tools to obtain the output using an API call to the one or more clinical software tools.

12. A computing system comprising:
one or more processors; and
one or more non-transitory computer-readable media storing instructions that are executable by the one or more processors to cause the computing system to perform operations, the operations comprising:
receiving an input query associated with a particular task domain of a plurality of clinical task domains;
obtaining a machine-learned prompt component and a curated prompt component, wherein the machine-learned prompt component comprises a plurality of machine-learned prompt values, and wherein the curated prompt component comprises a plurality of exemplar prompt values corresponding to one or more embedded natural language exemplars for the particular task domain from domain experts; and
generating an output responsive to the input query by processing a combined prompt and the input query using a pre-trained machine-learned model, wherein the combined prompt comprises the machine-learned prompt component and the curated prompt component.

13. The computing system of claim 12, wherein the operations comprise:
receiving the input query from a client device;
obtaining the machine-learned prompt component, the curated prompt component, or both, from a secure data store associated with the client device; and
returning the output to the client device.

14. The computing system of claim 13, wherein the machine-learned prompt component was learned in a secure environment based on training data associated with the client device.

15. The computing system of claim 14, wherein the machine-learned prompt component was learned on the client device.

16. The computing system of claim 12, wherein the curated prompt component is task domain specific, and wherein the machine-learned prompt component is shared across multiple task domains.

17. The computing system of claim 16, wherein the operations comprise:
receiving a different input query associated with a different task domain;
obtaining a different curated prompt component, wherein the different curated prompt component comprises one or more exemplars for the different task domain; and
generating a different output responsive to the different input query by processing the machine-learned prompt component, the different curated prompt component, and the different input query using the machine-learned model.

18. The computer-implemented method of claim 1, wherein the machine-learned model is configured to interact with one or more clinical software tools to obtain the output.

19. The computer-implemented method of claim 18, wherein the one or more clinical software tools comprise at least one tool selected from the following list: an electronic health record database, a data acquisition interface, medical image-processing software, patient communication software, biochemical simulation software, or insurance billing software.

20. One or more non-transitory computer-readable media storing instructions that are executable by one or more processors to cause a computing system to perform operations, the operations comprising:
receiving an input query associated with a particular task domain of a plurality of available task domains;
obtaining a machine-learned prompt component and a curated prompt component, wherein the machine-learned prompt component comprises a plurality of machine-learned prompt values for the plurality of available task domains, and wherein the curated prompt component comprises a plurality of exemplar prompt values corresponding to one or more embedded natural language exemplars for the particular task domain from domain experts; and
generating an output responsive to the input query by processing a combined prompt and the input query using a pre-trained machine-learned model, wherein the combined prompt comprises the machine-learned prompt component and the curated prompt component.
